SEQUENCE LISTING (2 sequences)

SEQ ID NO: 1 - DNA, 20 bp, synthetic construct
  source 1..20: mol_type = other DNA
tagctaggaagagtcacaca

SEQ ID NO: 2 - DNA, 79 bp, synthetic construct
  source 1..79: mol_type = other DNA
  misc_feature 1..24: note = DNA
  misc_feature 25: note = RNA
  misc_feature 26..79: note = DNA
  modified_base 25: mod_base = OTHER; note = 3'-Thiol adenosine
ttttttgtgtgactcttcctagctrtggttcgatcaagagatgtgcgtcttgatcgagacctgcgaccgtttttttttt

(19) United States
(12) Patent Application Publication
(10) Pub. No.: US 2025/0334540 A1
(43) Pub. Date: Oct. 30, 2025

(54) PORTABLE ELECTROCHEMICAL DEVICE FOR BIOSENSING AND METHODS OF MAKING AND USES THEREOF

(71) Applicant: McMaster University, Hamilton (CA)

(72) Inventors: Leyla Soleymani, Oakville (CA); Alexander Scott, Regina (CA)

(21) Appl. No.: 18/841,122

(22) PCT Filed: Feb. 24, 2023
(86) PCT No.: PCT/CA2023/050233

§ 371 Date: Aug. 23, 2024

Related U.S. Application Data
(60) Provisional application No. 63/314,003, filed on Feb. 25, 2022.

(51) Int. Cl. G01N 27/327; G01N 27/30; G01N 33/549

(52) U.S. Cl. CPC G01N 27/3276; G01N 27/301; G01N 33/549

(57) ABSTRACT

A portable biosensing readout system for analyzing a sample is described herein. The system include a biosensor comprising a working electrode, a reference electrode and a counter electrode. The system also includes a core potentiostat circuit coupled to the biosensor, the core potentiostat circuit having one or more amplifiers configured to inject an excitation signal into the biosensor; isolate the reference electrode; and convert current of an output signal from the biosensor to voltage. The system also includes a microcontroller unit in communication with the core potentiostat circuit, the microcontroller unit being configured to: generate various different types of excitation signals that are transmitted to the core potentiostat circuit and, subsequently, to the biosensor; receive the output signal from the core potentiostat circuit; and communicate the output signal to an external computing device. The system also includes a peripheral instrument in communication with the microcontroller unit for processing the sample.

### TECHNICAL FIELD

The present disclosure relates to biosensors, and in particular, to portable electrochemical and photoelectrochemical biosensing readout systems, methods of making and uses thereof.

### INCORPORATION OF SEQUENCE LISTING

A computer readable form of the Sequence Listing 3244-P67671 US01_SequenceListing.xml” (4,096 bytes), submitted via Patent Center and created on Feb. 28, 2025, is herein incorporated by reference.

### BACKGROUND

Biosensors are devices that bring together biorecognition and signal transduction elements for analyzing biologically-relevant analytes. These devices are ideally suited for use in point-of-care (PoC) diagnostics and health monitoring systems and are being extensively researched for diagnosing infectious diseases, cancers, cardiovascular diseases, and neurological diseases, to name a few. The rapid sample-to-result time of these devices at the point-of-need enables effective intervention by clinicians, which is expected to increase patient survivability and minimize the rates of disease transmission.

Since the commercialization of the first glucose biosensor in 1975, there has been tremendous interest in using electrochemical readout for disease management and health monitoring at the PoC. A number of electrochemical biosensors, which combine biorecognition elements with electrochemical transducers, have emerged in the research literature for detecting pathogens, extracellular vesicles, biomolecules, and small molecules, however, none of these classes of biosensors have penetrated the market to the same degree as PoC glucose monitors.

Multiple signal transduction strategies—electrochemical, photoelectrochemical, electronic, optical, and mechanical—have been widely used in biosensing. Among these, photoelectrochemical (PEC) signal transduction, combining optical excitation with electrochemical readout, has generated tremendous interest in recent years due to its low limit-of-detection (LOD), high sensitivity, and broad linear dynamic range. The decoupling of the mode of signal excitation from the signal readout reduces the background signals that are generated at high voltage biases, enhancing the signal-to-background ratio and limit-of-detection of PEC biosensors.

Issues related to sensor stability, trade-offs between assay time, sensitivity, and complexity, loss of performance in clinical samples, and integration challenges are all contributing factors to the difficulty in translating electrochemical biosensors from the laboratory to commercial markets. In the context of integration, it remains challenging, costly, and time consuming to adapt commercially-available electrochemical readers, potentiostatic or galavaostatic devices referred to generally as potentiostats, for use with specific PoC biosensors. In particular, commercially available potentiostats are prohibitively expensive and pose a number of developmental challenges for PoC analysis given their generally large size (although miniaturized potentiostats are becoming increasingly common), difficulty in use and data interpretation by non-experts, and perhaps most importantly their lack of actuation abilities needed for evaluating certain biological assays. A vast number of bioassays require sample heating, magnetic manipulation, and multiplexed measurements, which are typically performed using devices separate from the potentiostat, posing significant integration challenges.

In response, a number of lab-made potentiostats have been reported in the literature in recent years. While these devices are less costly than their commercial counterparts, they do little to address other key integration challenges that inhibit many electrochemical biosensors from penetrating the market. Many of these lab-made devices rely on a connection to a computer, which may not be feasible in all PoC applications, particularly in resource-limited environments. Others rely on highly technical software that is tailored to researchers, providing little instructions to the user. In addition, little work has been done to incorporate the aforementioned actuation instruments despite their incorporation in a number of electrochemical biosensing experiments. Lastly, both the commercial systems and the devices reported in the literature are designed for use with standard 2-electrode or 3-electrode systems and are therefore incompatible with multi-channel and multiplexed assays.

Despite increasing number of PEC biosensors reported in the literature (from 35 search results up to 2011 on Pubmed to an additional 764 results up to 2021), these devices are far from being utilized in real-world applications and are unavailable in commercial markets. In many cases, this can be attributed to the limited number of suitable photoactive materials that meet the stringent chemical stability requirements of biosensors and the challenges resulting from the poor photostability of common reagents and analytes (e.g. DNA probes, antibodies, antigens) under high energy optical excitation. Such issues are being addressed by researchers through the development of new chemically-stable photoactive materials such as semiconductor metal oxides, quantum dots and carbon-based nanomaterials and the use of photocurrent enhancing strategies such as creating hybrid plasmonic nanoparticle-metal oxide heterostructures, using carbon nanomaterials as highly conductive scaffolds, using organic ligands/dyes to improve optical absorption and dual sensitization via the coupling of large and small bandgap semiconductors heterostructures for exciting these materials at lower energies (i.e. visible wavelengths). The limited commercial success of PEC biosensors is further compounded by the challenges associated with employing existing PEC readout devices for PoC use. While PEC workstations are commercially available, they are both prohibitively expensive and are not portable. These devices are also feature rich, consisting of frequency analyzers, potentiostats, photodiode sensors, and tunable light sources. However, many of these features are needed for research measurements but are of no use in a PoC capacity. Conversely, none of the commercially available portable potentiostats support PEC biosensing, and their functionality cannot be expanded given their black-box nature. Excluding a recent PEC measurement system with surface mounted light emitting diodes controlled by a smartphone for optical excitation, none of the lab-made potentiostats reported in the literature can interface directly with optical-excitation sources to support PEC biosensing. Therefore, there is a need for a more robust, affordable, multiplexed, fully integrated portable system for PoC readout and actuation that can be controlled wirelessly.

The background herein is included solely to explain the context of the disclosure. This is not to be taken as an admission that any of the material referred to was published, known, or part of the common general knowledge as of the priority date.

### SUMMARY

According to a broad aspect, a portable biosensing readout system for analyzing a sample is described herein. The portable biosensing readout system includes a biosensor comprising a working electrode, a reference electrode and a counter electrode. The portable biosensing readout system also includes a core potentiostat circuit coupled to the biosensor, the core potentiostat circuit having one or more amplifiers configured to: inject an excitation signal into the biosensor; isolate the reference electrode; and convert current of an output signal from the biosensor to voltage. The portable biosensing readout system also includes a microcontroller unit in communication with the core potentiostat circuit, the microcontroller unit being configured to: generate various different types of excitation signals that are transmitted to the core potentiostat circuit and, subsequently, to the biosensor; receive the output signal from the core potentiostat circuit; and communicate the output signal to an external computing device. The portable biosensing readout system also includes a peripheral instrument for processing the sample, the peripheral instrument being in communication with the microcontroller unit to receive instructions from the microcontroller that control the peripheral instrument.

In at least one embodiment, the peripheral instrument is one or more of a heater, a magnetic module, an electromagnet and one or more tunable light sources.

In at least one embodiment, the output signal from the biosensor is an electrochemical signal or a photoelectrochemical signal.

In at least one embodiment, the microcontroller unit comprises an Arduino device that processes the output from the core potentiostat circuit.

In at least one embodiment, the microcontroller unit is coupled to a digital-to-analog converter and a reconstruction filter.

In at least one embodiment, the digital-to-analog converter comprises a Maxim Integrated MAX5217 16-Bit digital-to-analog-converter.

In at least one embodiment, the microcontroller unit is further configured to: generate a voltammetric excitation signal; compute a voltammetric excitation series; read the output signal received from the core potentiostat circuit; configure settings of various integrated circuits if the biosensing readout system; and communicate with the external computing device.

In at least one embodiment, the portable biosensing readout system further comprises: a dual output voltage reference; a multiplexer; and an analog-to-digital converter (ADC); and the plurality of amplifiers includes a control amplifier, a voltage follower, and a transimpedance amplifier.

In at least one embodiment, the voltage reference supports the ADC as the ADC requires a stable reference and biases the electrochemical cell (i.e., measurements are taken relative to the 1.5V reference rather than ground).

In at least one embodiment, the multiplexer is used to support experiments with one or two working electrodes.

In at least one embodiment, the ADC converts a raw voltage output of the core potentiostat circuit into a value that can be processed by the microcontroller.

In at least one embodiment, the multiplexer is configured to toggle between two or more working electrodes to perform sequential measurements of dual-signal assays or multi-channel assays.

In at least one embodiment, the multiplexer comprises a MAX4644EUT single-pole/double-throw (SPDT) switch.

In at least one embodiment, the analog-to-digital converter comprises a ADS122C04 24-Bit analog-to-digital converter.

In at least one embodiment, the plurality of amplifiers further comprises a dual operational amplifier, a precision operational amplifier, or a combination thereof.

In at least one embodiment, the dual operational amplifier comprises a AD8655/AD8656 low noise, precision CMOS amplifier.

In at least one embodiment, the precision operation amplifier comprises a LMP7721 3-Femtoampere input bias current precision amplifier.

In at least one embodiment, the dual output voltage reference comprises a REF20XX low-drift, low-power, dual-output, VREF and VREF/2 Voltage reference.

In at least one embodiment, the core potentiostat circuit may include a dual impedance converter/network analyzer chip, such as, but not limited to a AD5933 high precision impedance converter system.

In at least one embodiment, the core potentiostat circuit is configured to perform multiplexed measurements.

In at least one embodiment, the biosensor includes a biorecognition moiety immobilized onto a surface of the working electrode to recognize a presence of an analyte in the sample, the biorecognition moiety comprising a DNAzyme, an aptamer, an antibody, a nucleic acid, or a combination thereof.

In at least one embodiment, the peripheral instrument is a heater configured to heat the sample.

In at least one embodiment, the peripheral instrument is a magnetic module configured to perform magnetic manipulation of the sample.

In at least one embodiment, the peripheral instrument is an electromagnet configured to perform electromagnetic manipulation of the sample.

The portable biosensing readout system of claim 21, wherein the magnetic manipulation is used to isolate magnetic microbeads present in the sample.

In at least one embodiment, the peripheral instrument is a light source configured to optically excite the sample, the light source comprising: a light emitting diode (LED) matrix circuit comprising one or more LEDs.

In at least one embodiment, the LED matrix is powered by the microcontroller unit.

In at least one embodiment, the LED matrix is powered by a power source of the portable biosensing readout system.

In at least one embodiment, the one or more LEDs are a neutral white color and have a temperature greater than 3100 K and an intensity greater than 3000 mcd.

In at least one embodiment, the light source further comprises an ultraviolet light source.

In at least one embodiment, the LED matrix circuit has modifiable parameters for different assays, the modifiable parameters including on and off time and optical parameters, the optical parameters including LED intensity.

In at least one embodiment, the analyzing of the sample is by square wave voltammetry, cyclic voltammetry, linear sweep voltammetry, differential pulse voltammetry, normal pulse voltammetry, electrochemical impedance spectroscopy, chronoamperometry, or a combination thereof.

In at least one embodiment, the microcontroller unit is configured to control a total duration of a scan of the sample, control a voltage bias of the excitation signal, control a pulse amplitude of the excitation signal, control a pulse duration of the excitation signal and/or control a sampling rate of the excitation signal.

In at least one embodiment, the microcontroller unit is communicatively coupled to the external computing device by a Bluetooth Low Energy communication protocol, the microcontroller being configured to transmit data to the external computing device and receive instructions from the external computing device to enable wireless control of the portable biosensing readout system by the external computing device.

In at least one embodiment, the external computing device is a smartphone, a smartwatch, a personal digital assistant (PDA), a tablet computer or a computer.

The portable biosensing readout system of any one of claims 1 to 28, wherein the microcontroller unit is communicatively coupled to the external computing device by a wired connection.

In at least one embodiment, the portable biosensing readout system is a point-of-care device.

In accordance with a broad aspect, a method of analyzing a sample by a portable biosensing readout system, the method comprising: selecting one or more peripheral instruments to attach to a portable biosensing readout system; attaching the one or more peripheral instruments to the portable biosensing readout system; contacting the portable biosensing readout system with the sample to detect an output signal; and transmitting the output signal as data to an external computing device; and analyzing the data by the external computing device.

In at least one embodiment, the output signal is an electrochemical or a photoelectrochemical signal.

In at least one embodiment, the sample is a tissue sample, a cell culture isolate, blood, plasma, serum, cerebrospinal fluid, lymph, tears, urine, or saliva mucus.

In at least one embodiment, a biorecognition moiety on a biosensor of the portable biosensing readout system comprises DNAzymes, aptamers, antibodies, nucleic acids, or a combination thereof that is configured to recognize the presence of an analyte in the sample.

According to a broad aspect, the present application discloses a smartphone operated portable biosensing readout and actuating system that offers several key advantages over common laboratory based potentiostats that are typically single-plex and rely on non-integrated sample preparation or signal actuation devices.

This portable biosensing readout system is a fully integrated platform that is remotely controlled by a smartphone and offers the ability to perform standard voltammetric techniques with either standard three-electrode or dual signal assays to support multiple channel measurements and it also integrates peripheral instruments that may be used outside the research laboratory, for example at the PoC.

In accordance with an aspect, there is provided a portable biosensing readout system for measuring signals and performing a plurality of detection and measurement techniques, comprising: a) a power source; b) a microcontroller unit; c) a signal generation unit; d) a core potentiostat circuit; and e) interchangeable peripheral instruments.

In some embodiments, the signals are electrochemical and photoelectrochemical signals.

In some embodiments, the microcontroller unit comprises of circuitry in communication with said peripheral instruments to configure and control said peripheral instruments.

In some embodiments, the microcontroller unit comprises an Arduino device or related computer system that processes and reads the resulting output from the core potentiostat circuit.

In some embodiments, the signal generation unit comprises the microcontroller unit, a digital-to-analog converter, and a reconstruction filter.

In some embodiments, the digital-to-analog converter comprises a Maxim Integrated MAX5217 16-Bit digital-to-analog-converter.

In some embodiments, the microcontroller unit is responsible for, but not limited to generating a voltammetric excitation signal, computing the voltammetric excitation series, reading the resulting output from the electrochemical cell, configuring the settings of the various integrated circuits, and communicating with an external device.

In some embodiments, the core potentiostat circuit comprises a dual output voltage reference, a multiplexer, an analog-to-digital converter, and a plurality of amplifiers, such as, but not limited to a control amplifier, voltage follower, and a transimpedance amplifier.

In some embodiments, the multiplexer is used to toggle between two or more working electrodes to perform sequential measurements of dual-signal assays or multi-channel assays. In some embodiments, the multiplexer comprises a MAX4644EUT single-pole/double-throw (SPDT) switch.

In some embodiments, the analog-to-digital converter comprises a ADS122C04 24-Bit analog-to-digital converter.

In some embodiments, the plurality of amplifiers may comprise a dual operational amplifier, a precision operational amplifier, or a combination thereof.

In some embodiments, the dual operational amplifier comprises a AD8655/AD8656 low noise, precision CMOS amplifier.

In some embodiments, the precision operation amplifier comprises a LMP7721 3-Femtoampere input bias current precision amplifier.

In some embodiments, the dual output voltage reference comprises a REF20XX low-drift, low-power, dual-output, VREF and VREF/2 Voltage reference.

In some embodiments, the core potentiostat circuit may include a dual impedance converter/network analyzer chip, such as, but not limited to a AD5933 high precision impedance converter system.

In some embodiments, the core potentiostat circuit performs multiplexed measurements.

In some embodiments, the portable biosensing readout system interacts with a biosensor comprising a counter electrode, a working electrode, and a reference electrode.

In some embodiments, the portable biosensing readout system interacts with a biosensor that uses a biorecognition moiety immobilized onto the surface of the working electrode that to recognize the presence of an analyte in a given sample and comprises of but is not limited to a DNAzyme, aptamer, antibody, nucleic acid, or combinations thereof.

In some embodiments, the peripheral instruments include, but are not limited to heaters, magnetic modules, electromagnets, and tunable light sources.

In some embodiments, the peripheral instruments are used for sample heating, magnetic manipulation, and electromagnetic manipulation.

In some embodiments, magnetic manipulation is used to isolate magnetic microbeads.

In some embodiments, the peripheral light source uses an LED matrix circuit as an optical excitation source comprising: a) an LED matrix circuit comprising more than one LED light; b) a power source; c) LEDs that may be a neutral white color temperature greater than 3100K with an intensity greater than 3000mcd; d) a light source that may have ultraviolet capabilities; e) an LED matrix circuit that has modifiable parameters for different assays, such as, but not limited to LED on and off time and optical parameters, such as, but not limited to LED intensity.

In some embodiments, the plurality of detection and measurements techniques is selected from the group consisting of, but not limited to square wave voltammetry, cyclic voltammetry, linear sweep voltammetry, differential pulse voltammetry, normal pulse voltammetry, electrochemical impedance spectroscopy, chronoamperometry, and combinations thereof.

In some embodiments, the portable biosensing readout system has modifiable parameters such as, but not limited to the total duration of the scan, the voltage bias, pulse amplitude, pulse duration, and the sampling rate.

In some embodiments, the portable biosensing readout system utilizes a Bluetooth Low Energy communication protocol to enable the wireless control of said portable biosensing readout system and the transmission of generated data to an external device.

In some embodiments, the portable biosensing readout system interacts with an external device such as, but not limited to a smartphone, smartwatch, personal digital assistant (PDA), tablet computer, computer, or any wearable technology.

In some embodiments, the portable biosensing readout system communicates with an external device through a wired connection.

In some embodiments, the portable biosensing readout system is used as a point of care device.

The present application also provides a method of measuring signals within a sample using the portable biosensing readout system by selecting peripheral instruments to attach to portable biosensing readout system, attaching said peripheral instruments to portable biosensing readout system, contacting the portable biosensing readout system with the sample to detect signals, and reading the analyzed data on an extemal device.

In some embodiments, the method comprises measuring electrochemical or photoelectrochemical signals.

In some embodiments, the method comprises measuring a signal within a biological sample that includes at least one of a tissue sample, a cell culture isolate, blood, plasma, serum, cerebrospinal fluid, lymph, tears, urine, and saliva mucus.

In some embodiments, the method comprises the use of a biosensor interacting with the portable biosensing readout system that contains a biorecognition moiety on the biosensor and comprises of, but not limited to a DNAzyme, aptamer, antibody, nucleic acid, and combinations thereof that is able to recognize the presence of the analyte.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### BRIEF DESCRIPTION OF THE DRAWINGS

Certain embodiments of the disclosure will now be described in greater detail with reference to the attached drawings in which:

FIG. 1A shows a sample voltammetric excitation waveforms for square wave voltammetry scan type.

FIG. 1B shows a sample voltametric excitation waveform for cyclic voltammetry.

FIG. 1C shows a sample voltametric excitation waveform for linear sweep voltammetry.

FIG. 1D shows a sample voltametric excitation waveform for differential pulse voltammetry.

FIG. 1E shows a sample voltametric excitation waveform for normal pulse voltammetry.

FIG. 2A shows a photograph overview of a portable biosensing readout system and a 3D printed case, according to at least one embodiment described herein.

FIG. 2B is a functional block diagram of a portable biosensing readout system according to at least one embodiment described herein.

FIG. 2C is a pictorial of a smartphone application process flow.

FIG. 3A shows a cyclic voltammogram of different concentrations of a [Fe(CN)₆]⁴⁻/[Fe(CN)₆]³⁻ recorded using the portable biosensing readout system and screen printed electrodes with a scan rate of 200 mV/s comparison of cyclic voltammetry measurements between a portable biosensing readout system according to at least one embodiment described herein and a commercial device.

FIG. 3B shows a cyclic voltammogram of 2 mM [Fe(CN)₆]⁴⁻/[Fe(CN)₆]³⁻ recorded using the portable biosensing readout system and screen printed electrode at variable scan rates. The inset highlights the linear relationship that was observed between the peak current and the square root of the scan rate.

FIG. 3C shows a cyclic voltammogram of different concentrations of [Fe(CN)₆]⁴⁻/[Fe(CN)₆]³⁻ recorded using a commercial potentiostat and screen printed electrodes with a scan rate of 200 mV/s.

FIG. 3D shows a cyclic voltammogram of 2 mM [Fe(CN)₆]⁴⁻/[Fe(CN)₆]³⁻ recorded using a commercial potentiostat and screen printed electrode at variable scan rates. The inset highlights the linear relationship that was observed between the peak current and the square root of the scan rate.

FIG. 4A shows a top-down view of a sample heater peripheral device according to at least one embodiment described herein.

FIG. 4B is a graph showing a recorded surface temperature of the screen printed electrodes in the sample heater as a function of time.

FIG. 4C shows working principles of the bio-barcode assay. Probe DNA is deposited onto the surface of the WE. After depositing redox DNA, the screen printed electrodes are left to incubate for 30 minutes at 37° C. to facilitate hybridization with the probe.

FIG. 4D shows square wave voltammograms of the bio-barcode assay recorded using the portable biosensing readout system with screen printed electrodes before and after depositing: (i) 150 nM of target DNA and incubating for 30 minutes at approximately 37° C. using the portable sample heater peripheral; (ii) 150 nM of target DNA and incubating for 30 minutes at room temperature; (iii) blank target and incubating for 30 minutes at approximately 37° C. using the portable sample heater peripheral; (iv) blank target and incubating for 30 minutes at room temperature.

FIG. 5A shows a photograph of an electromagnet validation experimental setup.

FIG. 5B shows a photograph of a vial containing a mixture of DI water and magnetic beads before running the electromagnet for 30 minutes.

FIG. 5C shows a photograph of a vial containing a mixture of DI water and magnetic beads after running the electromagnet for 30 minutes.

FIG. 6A shows a schematic illustration of the two-electrode assay for an evaluation of the portable biosensing readout system for multiplexed analysis in exemplary embodiments of the disclosure. In the pre-target phase, the first electrode shows a large redox signal due to the redox tag on the DNAzyme, whereas the second electrode does not exhibit a signal. Following target incubation on Electrode 1, the DNAzymes are cleaved, and the redox DNA barcode, causing a decrease in the measured electrochemical current. The solution is then manually pipetted onto the second electrode. Following the incubation of the solution on the second electrode, the DNA barcode binding results in a large redox signal.

FIG. 6B shows square wave voltammetry as recorded using the portable biosensing readout system. (i) Electrode 1 measurements pre- and post-incubation with 10⁶ CFU/mL intracellular mixture of E. coli. (ii) Electrode 2 measurements pre- and post-incubation with the solution transferred from Electrode 1. (iii) Electrode 1 measurements pre- and post-incubation with a blank sample. (iv) Electrode 2 measurements pre- and post-incubation with a blank sample.

FIG. 6C is graphs showing fold changes for target and blank samples on (i) Electrode 1 and (ii) Electrode 2, target representing 10⁶ CFU/mL intracellular mixture of E. coli. and black representing PMT20.

FIG. 7A shows a block diagram representation of a portable biosensing readout system of at least one embodiment described herein, the system comprising a the Arduino Nano 33 BLE development board, digital-to-analog converter (DAC), a reconstruction filter, a core-potentiostat circuit comprising a voltage follower (VF), a control amplifier (CAmp), and a transimpedance amplifier (TIA), and an analog-to-digital converter (ADC) with a built-in low-pass filter (LPF).

FIG. 7B is a photograph of an LED matrix circuit according to at least one embodiment described herein.

FIG. 7C shows a pictorial of a smartphone application process flow, according to at least one embodiment described herein.

FIG. 8A is a graph showing anodic photocurrent generated by CA-TiO₂ photoelectrodes when bias voltages of 0.75 V and 1.0 V were applied relative to an Ag/AgCl reference electrode, where the inset illustrates photocurrent generation via oxidation of water.

FIG. 8B is a graph showing cathodic photocurrent generated by CA-TiO₂ photoelectrodes at bias voltages of −0.75 V and −1.0 V relative to an Ag/AgCl reference electrode where the inset illustrates photocurrent generation via reduction of oxygen species.

FIG. 9A is a graph showing results of a PEC cycling test conducted using a portable biosensing readout system according to at least one embodiment described herein.

FIG. 9B is a graph showing results of a PEC cycling test conducted using the Zahner CIMPS-QE/IPCE PEC workstation.

FIG. 9C is a graph showing results of a long term exposure test conducted using a portable biosensing readout system according to at least one embodiment described herein, where the inset shows the LED matrix circuit intensity measurement by recording the voltage drop across a photocell.

FIG. 9D is a graph showing results of a long term exposure test using the Zahner CIMPS-QE/IPCE PEC workstation where the inset illustrates photocurrent generation via the oxidation of ascorbic acid following TiO₂ photoexcitation.

FIG. 10A shows unmodified TiO₂, CA-modified TiO₂, probe DNA and target DNA.

FIG. 10B shows a DNA detection scheme. Following hybridization with target DNA, photocurrent decreases due to steric hinderance in the presence of ascorbic acid.

FIG. 10C is a graph showing photocurrent densities at unmodified, after-probe and after-target stages of signal-OFF DNA biosensor operation when a 0V bias was applied to the PEC cell relative to an Ag/AgCl reference electrode. The after-target samples include: only non-complementary target in buffer (NC), non-complementary target & complementary target in buffer (Target+NC), only human blood plasma (Plasma), complementary target in human blood plasma (Target+Plasma), non-complementary target in human blood plasma (Plasma+NC), and both non-complementary target & complementary target in human blood plasma.

FIG. 10D is a graph showing a summary of photocurrent signal decrease at each stage of the signal-OFF DNA biosensor operation.

FIG. 11A shows illuminated chronoamperometry experiments using a portable biosensing readout system accrding to at least one embodiment described herein with a Faraday cage.

FIG. 11B shows illuminated chronoamperometry experiments using the portable biosensing readout system accrding to at least one embodiment described herein without a Faraday cage.

FIG. 12 shows a graph of illuminated chronoamperometry experiments using a portable biosensing readout system according to at least one embodiment herein with an adjustable LED matrix circuit through variable PWM duty cycling using bare CA-TiO₂ photoelectrodes in exemplary embodiments of the disclosure.

FIG. 13A is a graph showing illuminated chronoamperometry experiments with different DNA target hybridization on the photoelectrode measured using a portable biosensing readout system according to at least one embodiment described herein.

FIG. 13B is a graph showing illuminated chronoamperometry experiments with different DNA target hybridization on the photoelectrode measured using a Zahner CIMPS-QE/IPCE PEC workstation in exemplary embodiments of the disclosure.

### DETAILED DESCRIPTION

### 1. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “sample” or “test sample” as used herein may refer to any material in which the presence or amount of a target analyte is unknown and can be determined in an assay. The sample may be from any source, for example, any biological (e.g. human or animal samples, including clinical samples), environmental (e.g. water, soil or air) or natural (e.g. plants) source, or from any manufactured or synthetic source (e.g. food or drinks). The sample may be comprised or is suspected of comprising one or more analytes.

The sample may be a “biological sample” comprising cellular and non-cellular material, including, but not limited to, tissue samples, urine, blood, serum, other bodily fluids and/or secretions.

It will be understood that any component defined herein as being included may be explicitly excluded by way of proviso or negative limitation, such as any specific compounds or method steps, whether implicitly or explicitly defined herein.

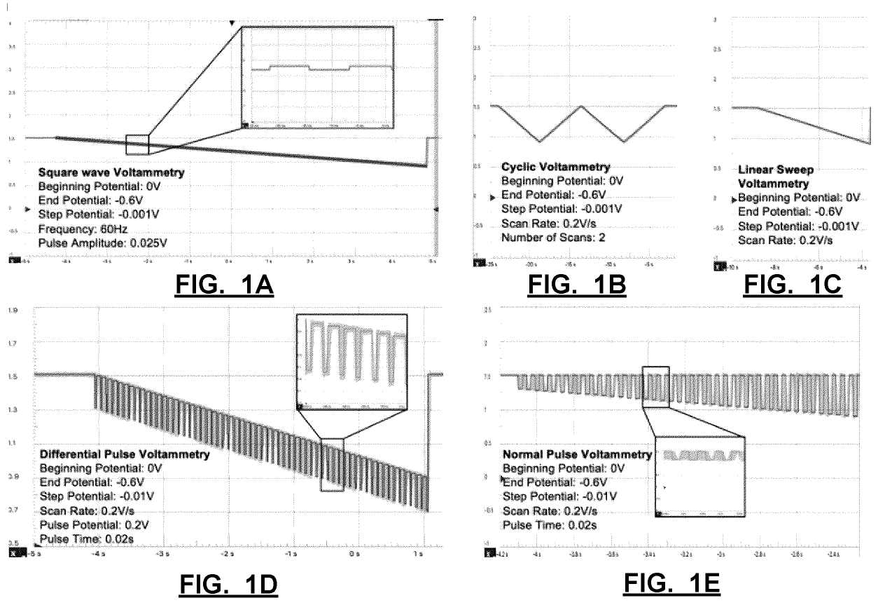

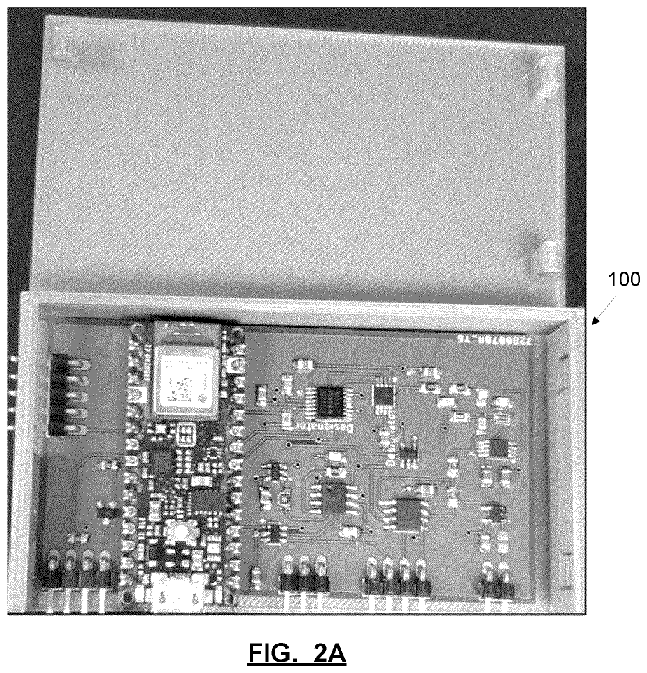

FIG. 1A shows a composite of sample voltammetric excitation waveforms for some of the supported voltammetric scan types in exemplary embodiments of the disclosure. FIG. 1A shows square wave voltammetry; FIG. 1B shows cyclic voltammetry;

FIG. 1C shows linear sweep voltammetry; FIG. 1D shows differential pulse voltammetry; and FIG. 1E shows normal pulse voltammetry. Scans were recorded using a Digilent Analog Discovery USB oscilloscope. Note, a 1.5V offset is applied to the excitation signals given that the Arduino Nano 33 BLE can only output values between 0V-3.3V. This offset is accounted for by applying a 1.5V bias to the non-inverting terminals of both the control amplifier and transimpedance amplifier.

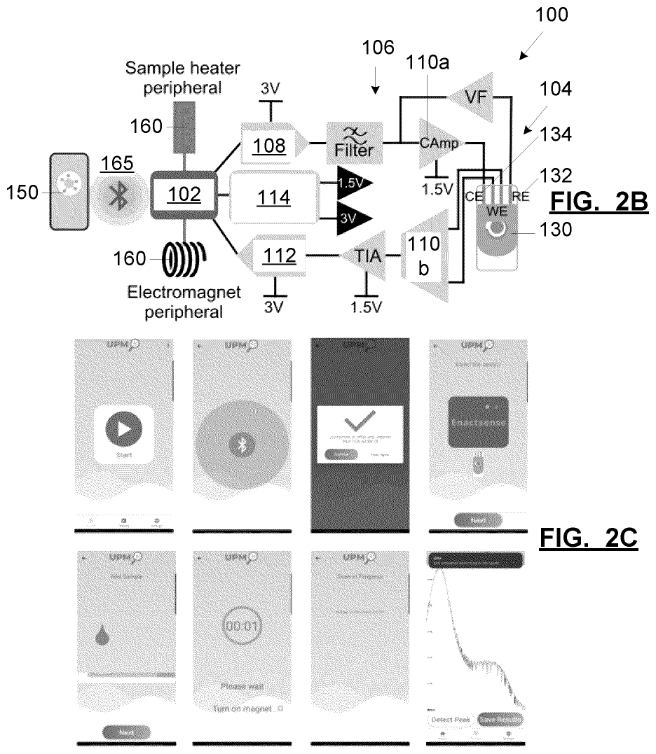

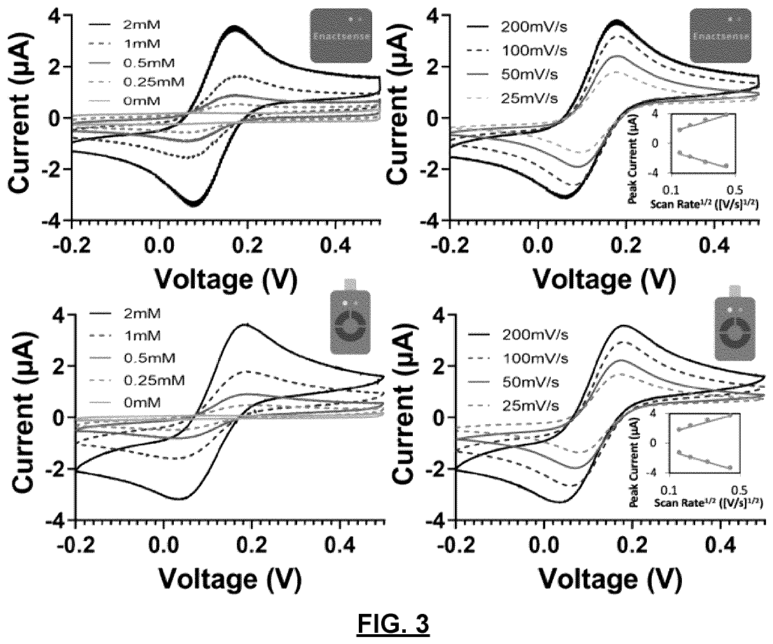

FIG. 2A shows a photograph of, and FIG. 2B shows a schematic diagram of, a portable biosensing readout system 100, according to at least one embodiment described herein. The portable biosensing readout system 100 comprises a microcontroller unit (e.g., an Arduino development board) 102, a biosensor 104, a potentiostat circuit 106 having, optionally, a digital-to-analog converter (DAC) 108, one or more operational amplifiers (op-amps) 110i, an analog-to-digital converter (ADC) 112, and a voltage reference 114.

Portable biosensing readout system 100 is configured to couple to one or more peripheral instruments 120.

Biosensor 104 includes a working electrode 130, a reference electrode 132 and a counter electrode 134.

Core potentiostat circuit 106 is coupled to the biosensor 104. Core potentiostat circuit 106 has one or more amplifiers 110i configured to: inject an excitation signal into the biosensor 104; isolate the reference electrode 132; and convert current of an output signal from the biosensor 104 to voltage.

Microcontroller unit 102 is in communication with the core potentiostat circuit 106. Microcontroller unit 102 is configured to generate various different types of excitation signals that are transmitted to the core potentiostat circuit 106 and, subsequently, to the biosensor 104. Microcontroller unit 102 is configured to receive the output signal from the core potentiostat circuit 106 and communicate the output signal to an external computing device 150. Microcontroller unit 102 is configured to communicate with a peripheral instrument 160 for processing the sample. Peripheral instrument 160 is in communication with the microcontroller unit 102 to receive instructions from the microcontroller until 102 that control the peripheral instrument 160.

For example, in at least one embodiment, peripheral instrument 160 may include a heater. In at least one embodiment, heater 160 may be used to heat a sample being tested using the portable biosensing readout system 100.

For example, in at least one embodiment, peripheral instrument 160 may include a magnetic module. In at least one embodiment, magnetic module 160 may be used for magnetic manipulation of, for example, one or more components of the sample being tested using the biosensing readout system 100. For example, in some embodiments, the magnetic manipulation may be used to isolate magnetic microbeads present in the sample being tested using the biosensing readout system 100.

For example, in at least one embodiment, peripheral instrument 160 may include one or more electromagnets. In at least one embodiment, electromagnet(s) 160 may be used for electromagnetic manipulation of, for example, one or more components of the sample being tested using the biosensing readout system 100.

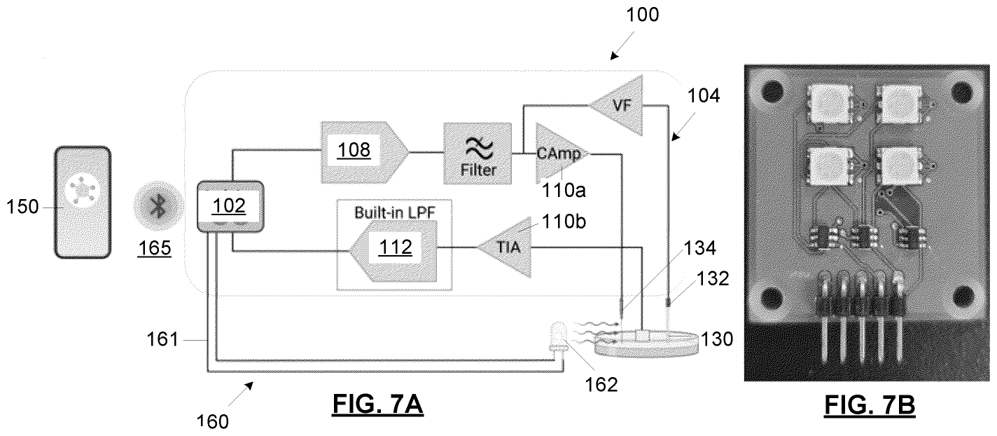

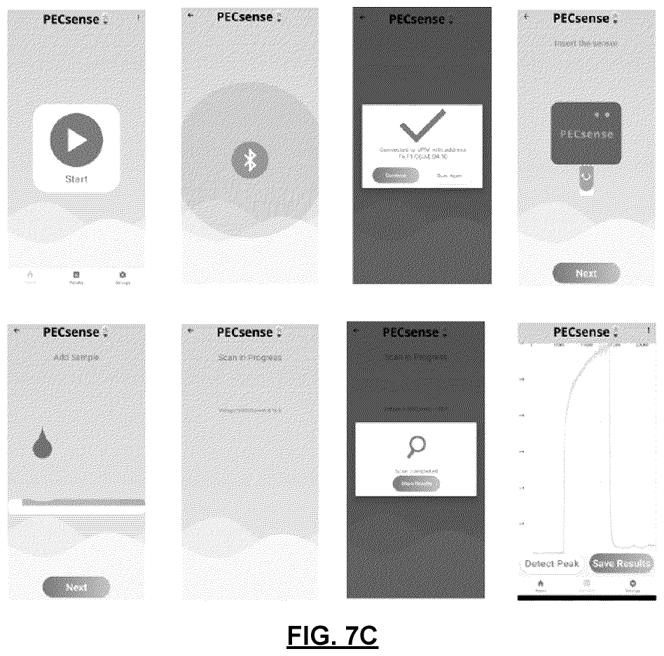

For example, in at least one embodiment, for example shown in FIG. 7A, peripheral instrument 160 may include one or more tunable light sources, or light sources, such as but not limited to a light emitting diode (LED). In at least one embodiment, the peripheral light source 160 may use an LED matrix circuit as an optical excitation source comprising: a) an LED matrix circuit 161 comprising more than one LED light 162; b) a power source 163; c) LEDs 162 that may be a neutral white color temperature greater than 3100K with an intensity greater than 3000mcd; d) a light source 164 that may have ultraviolet capabilities; e) an LED matrix circuit 161 that has modifiable parameters for different assays, such as, but not limited to LED on and off time and optical parameters, such as, but not limited to LED intensity.

In at least some embodiments, portable biosensing readout system 100 may operate as both an electrochemical reader and an actuator. Portable biosensing readout system 100 is communicatively coupled to a computing device 150, for example a smartphone, having an application stored thereon for processing data received from the potable biosensing readout system 100.

In at least one embodiment, portable biosensing readout system 100 is communicatively coupled to the computing device 150 by a wireless network 165, such as any wireless network 165 known to those skilled in the art, including but not limited to a Wifi network, a Bluetooth network, such as but not limited to a Bluetooth Low Energy (BLE) communication protocol.

Computing device 150 is configured to provide instructions to portable biosensing readout system 100 that can remotely control the portable biosensing readout system 100.

### Examples

### Example 1: Portable Electrochemical Reader

### Materials and Methods

Reagents and Materials: Escherichia coli K12 (E. coli K12; MG1655), which is regularly maintained in one of McMaster University's laboratory, was used in this study. Lyophilized oligonucleotides were purchased from Integrated DNA Technologies, Inc (Iowa, United States). Methylene blue N-Hydroxysuccinimide (NHS) esterwas purchased from Glen Research (Virginia, United States). Dimethyl sulfoxide (DMSO), sucrose, xylene cyanole FF, bromophenol blue, 10× tris-borate-EDTA buffer (TBE), tris, ethylenediaminetetraacetic acid (EDTA), Sodium Acetate, Glacial Acetic Acid, and sodium chloride was purchased from Sigma-Aldrich (Ontario, Canada). Urea, and 40% 29:1 bis/acrylamide was purchased from Bioshop Canada (Ontario, Canada). A Stuart handheld ultraviolet (UV) lamp 254/365 nm was purchased from Cole-Parmer (Ontario, Canada). 0.2 μm filter discs were purchased from Acrodisc. The shaker (76407108) was purchased from VWR (Ontario, Canada). BTV-AC1 electrochemical sensors (referred to here on as on-chip electrodes) were purchased from Palmsens BV (Netherlands). Magnetic beads, which are regularly maintained in one of McMaster University's laboratory, were used to demonstrate the portable biosensing readout system's capability of performing basic magnetic manipulation. All other chemicals were purchased from Sigma-Aldrich and were used without further purification. The specific DNA sequences used in this work can be found in Table 1 below:

TABLE 1
Specific DNA sequences used

| | | | |
|---|---|---|---|
| TP | 3′-Thiol | TAGCTAGGAAGAGTCACACA | Capture DNA probe |
| (20 nt) | | (SEQ ID NO: 1) | |
| TD | 5′-Methylene | MBTTTTTTGTGTGACTCTTCCTAGCT | Ecoli DNAzyme |
| 79 nt | blue; | RTGGTTCGATCAAGAGATGTGCGTC | |
| | R = riboA; 3′- | TTGATCGAGACCTGCGACCGTTTTTT | |
| | Thiol | TTTT SEQ ID NO: 2) | |

Device Engineering: The device presented in this disclosure, was designed around the low amplitude signal constraints associated with typical biosensing experiments and is capable of performing several types of voltammetric functions (as seen in FIGS. 1A-1E). These individual functions were developed for each of the supported voltammetric techniques. Timer interrupts, of which the specific timing intervals are determined in accordance with the scan's timing parameters, are used such that the output to the DAC can be appropriately updated. To minimize input referred noise associated with the ADC, the minimum possible sampling rate is used based on the timing characteristics of the voltammetric scan. For square wave voltammetry, this is dependent on the frequency. For all other supported voltammetric techniques, the minimum ADC sampling rate is determined based on the scan rate.

The portable biosensing readout system is controlled via an Android application through Bluetooth Low Energy (BLE) rather than a dedicated program on a desktop computer, thereby capitalizing on the widespread global adoption, increasing processing power, and the decreasing cost of smartphones, and also rendering the system more portable and accessible. A summary of some of the device's capabilities can be found in Table 2 below:

TABLE 2
Summary of device capabilities

| | |
|---|---|
| Supported Voltametric Scan | Square wave voltammetry, cyclic |
| types | voltammetry, linear sweep, differential pulse, |
| | normal pulse |
| Current range | −10 μA to 10 μA (−20 μA to 20 μA for |
| | differential methods) |
| Supply voltage | 4.5-21 V |
| Signal generation resolution | 16-bit |
| Measurement resolution | 24-bit (20-bit effective) |
| Measurement sampling rate | Programmable from 20sps-2ksps |
| Output range | −1.5 V to 1.5 V |

Device schematics and printed circuit boards (PCBs) were designed using Autodesk Eagle. PCB manufacturing was conducted by JLCPCB and the individual components were hand-soldered. The portable biosensing readout system is composed of the Arduino Nano 33 BLE development board, the MAX5217 digital-to-analog converter (DAC), a reconstruction filter made of the AD8656 dual operational amplifier, a core potentiostat circuit consisting of the AD8606 dual operational amplifier and the LMP7721 precision operational amplifier, the ADS122C04 analog-to-digital converter (ADC), and the dual output REF2030 voltage reference.

In at least one embodiment, the portable biosensing readout system incorporates, or connects to, peripheral devices that expand upon the device's capabilities. A custom sample heater circuit was developed to facilitate away from lab sample heating. A 3D printed case made of polylactic acid was designed using Autodesk Inventor and printed using an Original Prusa i3 MK3S 3D printer to house an adhesive flexible polyimide heater. This heater was made by Icstation and was purchased through Amazon Canada. The KS0320 Keyestudio Electromagnet Module was also integrated to facilitate away-from-lab magnetic manipulation and was purchased through Amazon Canada.

The firmware was developed using the Arduino integrated development environment and it is responsible for computing the voltammetric excitation series, reading the resulting output from the electrochemical cell, controlling the peripheral devices, configuring the settings of the various integrated circuits, and communicating with the smartphone. The ArduinoBLE external library was used to simplify BLE communications. Sparkfun's ADS122C04 Arduino library was used to interface with the ADC and properly configure its registers. Individual functions were created for each of the supported voltammetric scan types.

An accompanying smartphone application was developed in Java using the Android Studio integrated development environment. This application is responsible for remotely controlling the portable biosensing readout system, providing means of user interface, and performing any necessary signal processing and analysis. The BlessedBLE library was used to facilitate communication with the portable biosensing readout system. The voltammetric scans are presented in graphical format using the open-source MPAndroidChart graphing library. In-app data smoothening was conducted using Marcin Rzeinicki's open-source SGFilter Java class.

Electrical and Electrochemical Characterization: To quantitatively assess the portable biosensing readout system's noise performance, the electrode connections were left open, and the current was measured in a typical laboratory environment for 7 minutes in order to measure the input-referred noise of the current sensing portion of the portable biosensing readout system (transimpedance amplifier and ADC). The ADC sampling rate was set to 20 samples per second (sps). No efforts were taken to shield the device from any forms of interference.

Subsequently, the redox behavior of [Fe(CN)₆]⁴⁻/[Fe(CN)₆]³⁻ was investigated at various concentrations and scan rates. On-chip electrodes were cleaned by performing 30 cyclic voltammetry scans with 0.5M sulfuric acid from 0-1.5V in 0.001V steps with a scan rate of 0.1V/s using a commercial potentiostat. Following this, 2 mM, 1 mM, 0.5 mM, 0.25 mM, and 0 mM of the [Fe(CN)₆]⁴⁻/[Fe(CN)₆]³⁻ solutions were prepared in 25 mM phosphate buffer saline (PBS) and 25 mM NaCl buffer (25:25 buffer). 40tL drops of the solutions were individually pipetted onto the on-chip electrodes following washing with deionized water. Cyclic voltammetry was then performed for a potential range of −0.2V—0.5V, with steps of 0.001V and a scan rate of 0.2V/s. Subsequently, 40tL drops of the 2 mM [Fe(CN)₆]⁴⁻/[Fe(CN)₆]³⁻ solution prepared in 25:25 buffer were pipetted onto new on-chip electrodes. Cyclic voltammetry was then performed from −0.2V—0.5V, with steps of 0.001V and scan rates of 0.2V/s, 0.1V/s, 0.05V/s, and 0.025V/s. In-app data smoothening was not performed. The performance of the portable biosensing readout system was compared to the commercially available Sensit Smart.

Peripheral Devices Validation: The performance and characteristics of the sample heater and electromagnet were investigated. After first allowing the sample heater to pre-heat for one minute, an on-chip electrode was placed inside the sample heater for 30 minutes. The surface temperature of the on-chip electrode was then recorded every 5 minutes using the SOVARCATE 960 infrared thermometer. Following this, new on-chip electrodes were cleaned by performing 30 cyclic voltammetry scans with 0.1M sulfuric acid from 0 to 1.5V in 0.001V steps with a scan rate of 100 mV/s using a commercial potentiostat. A mixture of 1tM tris(2-carboxyethyl)phosphine (TCEP) (1:100) and 1tM probe was prepared in 25 mM phosphate buffer saline (PBS), 25 mM NaCl, and 100 mM MgCl2 buffer 25:25:100 solution. After allowing this mixture to stabilize following a 2-hour incubation at room temperature, 3tL drops of the mixture were pipetted onto the on-chip electrodes. The on-chip electrodes were then left to incubate at room temperature in a humid environment for 18 hours. Following this incubation period, 3tL drops of 100 mM mercapto hexanol (MCH) backfill were pipetted onto the on-chip electrodes and left to incubate for 20 minutes at room temperature. Square wave voltammograms were recorded using the portable biosensing readout system from 0V to −0.6V in 0.001V steps with a frequency of 60 Hz and a pulse amplitude of 0.025V. Subsequently, 10tL of methylene blue tagged target DNA with a concentration of 150 nM was pipetted onto half of the on-chip electrodes and 10tL of PMT20 was pipetted onto the remaining on-chip electrodes. The on-chip electrodes were then incubated for 30 minutes at approximately 37° C. using the sample heater. Following this incubation, square wave voltammograms were recorded using the portable biosensing readout system from 0V to −0.6V in 0.001V steps with a frequency of 60 Hz and a pulse amplitude of 0.025V. A similar procedure was performed with another set of on-chip electrodes but they were instead incubated at room temperature (RT) during the target DNA hybridization step. In-app baseline subtraction and data smoothening were performed.

In order to demonstrate the portable biosensing readout system's ability to control an electromagnet within an electrochemical biosensing context, it was used to isolate magnetic microbeads in a suspension. The electromagnet was turned on and was placed directly beside a rectangular vial containing 14 tL of DI water and 1 tL of magnetic microbeads for 30 minutes.

Evaluation of a Two-Working Electrode Assay: In accordance with the manufacturer protocol, 5′-Amino-modified E. coli DNAzyme (D) was labeled using methylene blue NHS Ester diluted in DMSO. The lyophilized DNAzyme was diluted in 0.1 M Carbonate/Bicarbonate buffer (pH 9). Next, the methylene blue NHS Ester was added to the DNAzyme for methylene blue tagging and left to incubate for two hours at room temperature. Subsequently, the DNAzyme was purified using 10% urea 40% 29:1 Bis/Acrylamide page gel. Prior to loading into the gel, an Ethanol precipitation (0.1× Sodium acetate (pH=5.2), 2.5× 100% ethanol) step was performed. The gel was run for 1 hour and the DNAzyme bands were vitalized and cut using UV light (240 nm). Afterwards, the gel was crushed and eluted using an in-house elution buffer (200 mM NaCl, 10 mM Tris pH=7.5, 1 mM EDTA). The crushed gel was eluted two more times on a heated shaker at 300 rpm at 37° C. for 30 minutes. A final ethanol precipitation step was applied. The retrieved DNAzyme was then diluted in RNA/DNA free waterforfurther use.

The crude intracellular matrix (CIM) preparation protocol was adapted. Escherichia coli K12 (MG1655) was grown under the appropriate conditions and cultured in lysogeny broth (LB) media overnight until optical density reached OD600˜1.0. Subsequently, 1 mL of each bacterial culture was centrifuged at 10,000 g for 10 minutes and the clear supernatant was discarded. The cells were then suspended in 500 μL of 1× reaction buffer (50 mM HEPES, 150 mM NaCl, 15 mM MgCl2, Tween 20 0.01%, pH 7.5).

The cell suspension was heated at 90° C. for 5 minutes and subsequently left at room temperature for an additional 10 minutes to ensure proper cell lysis. Next, the suspension was centrifuged at 13,000 g for 10 minutes. The clear supernatant was then collected and passed through a 0.2 μm filter disc. The supernatant was aliquoted and stored at −20° C. and was used in DNAzyme cleavage experiments as needed. This CIM supernatant corresponds to ˜2 Å˜ 10⁹ cells/mL.

On-chip electrodes were cleaned by performing 10 cyclic voltammetry scans with 0.1 M sulfuric acid from 0-1.5V in 0.001V steps with a scan rate of 100 mV/s using a commercial potentiostat. Next, 3 tM of thiolated probe (TP) was reduced using 300 tM TCEP (1:100) for 2 hours in the dark at room temperature. Concurrently, 5 tM of thiolated DNAzyme (TD) was reduced using 500tM TCEP (1:100) for 2 hours in dark at room temperature. After this reduction time had passed, 3tL drops of the TP and TD were deposited onto the respective electrodes. The electrodes were then left to incubate at room temperature for 18 hours. Following this incubation period, the on-chip electrodes were washed in 25:25 buffer. 3 tL drops of 100 mM MCH backfill were deposited onto the on-chip electrodes and left to incubate for 20 minutes in the dark at room temperature. Square wave voltammetry was performed using the portable biosensing readout system from 0V to −0.6 V in 0.001 V steps with a frequency of 60 Hz and a pulse amplitude of 0.025 V. Subsequently, a 10 tL solution of the aforementioned 10⁶ CFU/mL bacterial CIM were pipetted onto half of the available on-chip electrodes assigned as Electrode 1 and 10 tL of PMT20 was pipetted onto the remaining on-chip electrodes assigned as Electrode 1 on-chip electrodes. These on-chip electrodes were then incubated for 30 minutes at 37° C. in a conventional laboratory oven. Following this 30-minute incubation, the solution on top of these on-chip electrodes were manually transferred to the on-chip electrodes assigned as Electrode 2. The on-chip electrodes assigned as Electrode 2 were then incubated for 30 minutes at 37° C. in a conventional laboratory oven. Square wave voltammetry was recorded using the portable biosensing readout system from 0V to −0.6 V in 0.001 V steps with a frequency of 60 Hz and a pulse amplitude of 0.025 V. In-app baseline subtraction and data smoothening were performed.

### Results and Discussion

Device Engineering: A miniaturized (75 mm by 40 mm) and inexpensive (˜95 USD) electrochemical reader and actuator was developed that includes an accompanying smartphone application, specifically made for use with biological assays. The Bluetooth Low Energy (BLE) communication protocol, a recent low-power revision of the traditional Bluetooth communication scheme, is employed so that the smartphone can remotely control the portable biosensing readout system. Accordingly, a universal device was developed that can interface with a wide range of smartphones regardless of their make or model.

The portable biosensing readout system (FIGS. 1A and 1B) is composed of the Arduino Nano 33 BLE development board, a digital-to-analog converter (DAC), various operational amplifiers (op-amps), an analog-to-digital converter (ADC), and a voltage reference. The Arduino Nano 33 BLE development board was used as it natively supports BLE communications without the need for an external BLE module. Furthermore, the Arduino Nano 33 BLE development board has a number of programmable input and output channels, which was used to communicate with the DAC, ADC, sample heater, and electromagnet. Finally, this board is compatible with a number of open-source libraries, which facilitated firmware development. Since the Arduino Nano 33 BLE does not output analog voltages, an external DAC was added. Specifically, the MAX5217 16-bit DAC was utilized to convert the signal from the Arduino Nano 33 BLE development board into an analog voltammetric excitation signal, which controls the potential between the reference electrode (RE) and the working electrode (WE). The dual output REF2030 voltage reference is used to set the maximum voltage output of this DAC to 3V. Accordingly, the voltage resolution of the DAC is 46 μV, which is well below the step potential required for most voltammetric scans used in biosensing and is in line with many commercial potentiostats. A reconstruction filter follows the output of the DAC in order to attenuate image frequencies, thereby correcting for the staircase effect associated with the discrete nature of DACs and leading to the generation of smooth voltammetric excitation signals.

The core potentiostat circuit, composed of three op-amps, is situated after the reconstruction filter. The first op-amp is a voltage follower (VF) which is used to isolate and prevent the flow of current through the RE, thereby ensuring the RE can provide a stable reference. With that said, real-world op-amps do not have an infinite input impedance meaning that an input bias current will still flow through the RE. Next, a control amplifier (CAmp) is responsible for injecting current into the cell to compensate for the redox reaction occurring at the WE. Lastly, a transimpedance amplifier (TIA) converts the current output of the cell into a voltage. The AD8606 dual amplifier was used for the VF and CAmp in part due to its low input bias current (0.2 pA). The small input voltage offset (20 μV) and low voltage noise density (8 nV/Hz^{1/2}) of this op-amp also ensure that the applied potential is accurate. Whereas for the TIA, the LMP7721 was selected chiefly due to its markedly low input bias currents (3 fA), thereby ensuring that the current to voltage conversions are as accurate as possible. In order to support multiplexed measurements as required in many biosensing experiments, MAX4644EUT was added to toggle the WE that is connected to the input of the TIA.

Even though the Arduino Nano 33 BLE development board has a built-in ADC, the resolution does not meet the stringent requirements associated with many electrochemical biosensing experiments. As such, the external ADS122C04 ADC was used to convert the voltage output of the TIA into a digital signal that can be recorded by the Arduino Nano 33 BLE and later transmitted to the smartphone. This ADC has an effective 20-bit resolution, programmable sampling rate, and features a built-in low-pass filter to suppress 50/60 Hz line noise when sampling at 20 samples per second (sps).

The portable biosensing readout system was designed to interface with a portable heater and an electromagnet to support away-from-lab sample heating and magnetic manipulation. The heater is powered by a separate 12 V power supply and can reach a maximum temperature output of 170° C. The temperature of the sample heater and the magnetic field strength are controlled directly by the portable biosensing readout system.

In order to make the portable biosensing readout system accessible and easy-to-use, an accompanying Android application was developed that is responsible for connecting to the portable biosensing readout system, adjusting the voltammetric scan parameters, guiding the user through an experiment, signaling the portable biosensing readout system to begin a measurement, and displaying the results (as outlined in FIG. 2C). The BlessedBLE library was employed to facilitate communication with the portable biosensing readout system. Namely, this library was used to search for and connect to the portable biosensing readout system, to inform the portable biosensing readout system of any modifications made to the scan parameters, to receive scan measurements from the portable biosensing readout system, and to send instructions to the portable biosensing readout system in order to remotely control the peripheral instruments, in this case the sample heater or electromagnet. The voltammetric scan parameters can either be manually entered by the user or imported by scanning custom QR codes, which have been designed to contain embedded information associated with a specific electrochemical biosensor. Editable parameters include the scan type, beginning potential, end potential, step potential, among other scan type specific parameters. Several graphical animations were created to help walk the user through the various stages of a typical electrochemical experiment. These stages include connecting the on-chip electrodes to the portable biosensing readout system, adding the sample to the on-chip electrodes, heating the sample, and performing the scan. Preloaded video demonstrations provide the user with additional guidance. The MPAndroidChart open-source graphing library was used to generate graphical representations of scan measurement (voltammograms). However, the raw data can also be saved locally on the device in comma-separated values (CSV) format.

Another key responsibility of the Android application is to perform signal processing. Owing to the noise background of electrochemical measurements that can at times be significant, data can be obfuscated leading to analytical errors. To address this, various signal processing techniques have been proposed to smooth electrochemical datasets including moving median filters and Savitzky-Golay filters [86]. While moving median filters can be employed to help smooth the data, this approach can lead to truncated signal peaks. Conversely, Savitzky-Golay filters more accurately preserve the structural integrity of the original signal. As such, Marcin Rzeinicki's open-source SGFilter Java class was employed to perform this data smoothening. It should be noted that this approach does not address the fact that baseline currents, resulting from the composition of the WE, electrolyte, presence of dissolved oxygen, and experimental ambient conditions, can obscure the true peak amplitude of a signal. In order to remove the abovementioned baseline currents, a moving average baseline correction algorithm was developed. This algorithm computes the moving average baseline and subtracts this curve from the raw signal. The last analytical function developed for the smartphone application is a simple peak detection algorithm that returns a list of local maxima and minima.

From a firmware perspective, two external libraries were used: the ArduinoBLE library which is used to facilitate BLE communication, and Sparkfun's ADS122C04 Arduino library which is used to interface with the ADC and properly configure its registers. A function was developed to automatically reconfigure the sampling rate of the ADC based on the timing parameters of the voltammetric scan, allowing for improved signal-to-noise ratios when performing slower voltammetric scans. Individual functions were created for each of the supported voltammetric scan types. Timer interrupts are used to appropriately update the DAC output and poll from the ADC in accordance with the scan's timing parameters. In order to support biosensing experiments with dual signal electrodes, the firmware toggles the MAX4644EUT so that sequential electrochemical measurements can be performed.

Electrical and Electrochemical Characterization: The noise performance, as well as the electrochemical performance of the portable biosensing readout system, were investigated and compared to the commercially available Sensit Smart, a widely-used miniaturized potentiostat by PalmSens. Electrochemical biosensing experiments are susceptible to electrical noise given their low signal amplitude and/or high frequency. This unwanted distortion of the output signal can be generated intrinsically through the electrical components of the potentiostat or coupled from an external source. Open circuit noise measurements were performed to quantify the input-referred noise of the portable biosensing readout system. It was found that the standard deviation of the open-circuit noise was greater than the effective resolution of the ADC (36 pA versus 19 pA). As such, the current sensing abilities of the portable biosensing readout system are limited by the input-referred noise of the device rather than the resolution of the ADC itself.

To understand the electrochemical performance of the portable biosensing readout system, cyclic voltammetry was performed using the [Fe(CN)6]4−/[Fe(CN)6]3− redox couple at five different concentrations (2 mM, 1 mM, 0.5 mM, 0.25 mM, and 0 mM) with a scan rate of 200 mV/s. Based on this scan rate, the ADC sampling rate for the portable biosensing readout system was automatically set to 600 sps. The performance of the portable biosensing readout system was compared to the commercially available Sensit Smart. The resulting voltammograms as recorded by the portable biosensing readout system and Sensit Smart can be found in FIG. 3A and FIG. 3C respectively.

Using a concentration of 2 mM of [Fe(CN)6]4−/[Fe(CN)6]3−, cyclic voltammetry was performed at four different scan rates (200 mV/s, 100 mV/s, 50 mV/s, and 25 mV/s). This corresponds to sampling rates of 600 sps, 175 sps, 90 sps, and 45 sps, which were automatically determined by the firmware. The resulting voltammograms as recorded by the portable biosensing readout system and Sensit Smart can be found in FIG. 3B and FIG. 3D respectively. The cyclic voltammograms show a linear change in peak current with respect to the square root of scan rate (Inset-FIG. 3C-D) demonstrating the expected diffusion-controlled behaviour.

The peak currents and voltages and peak shapes as recorded by the portable biosensing readout system, and the commercial device are similar. Differences can partly be attributed to variation between the individual on-chip electrodes. It should be noted, however, that the noise performance of the portable biosensing readout system improved when lower ADC sampling rates were used, hence why the high scan rate datasets appear noisier in comparison to the commercial device. Given the black-box nature of the commercial device, it is unknown if any signal filtration or processing is automatically conducted post-data collection. Data smoothening was not applied to the portable biosensing readout system's datasets.

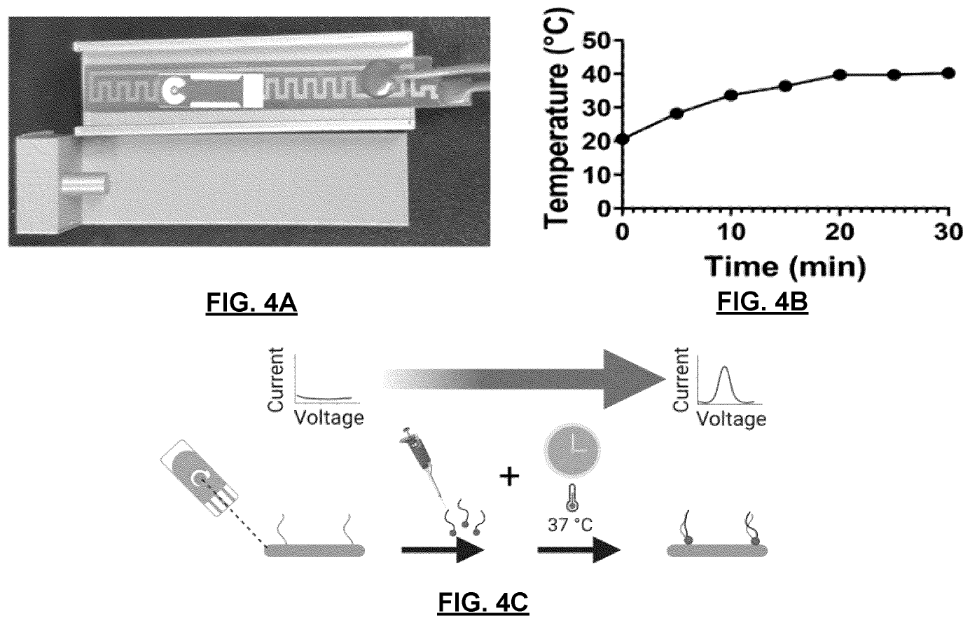

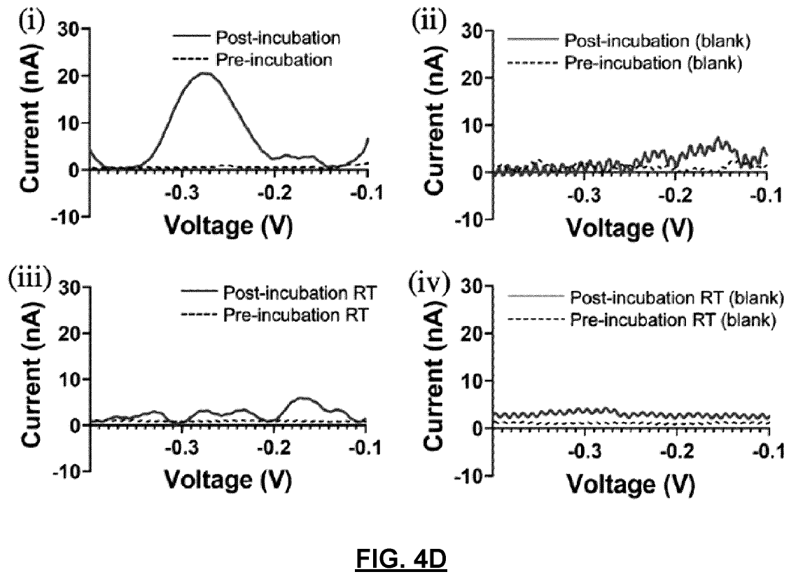

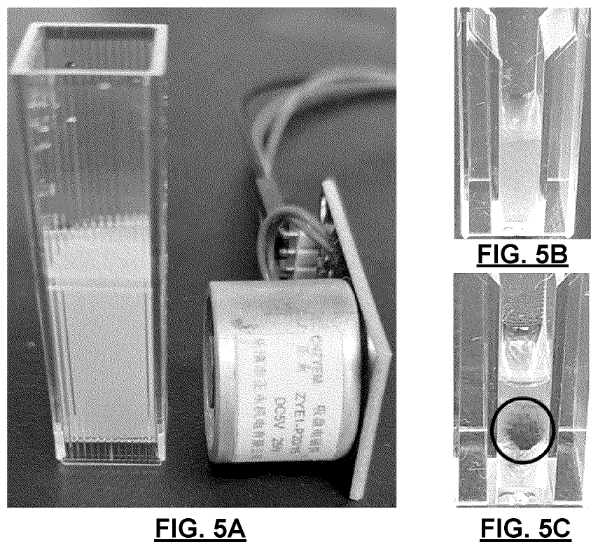

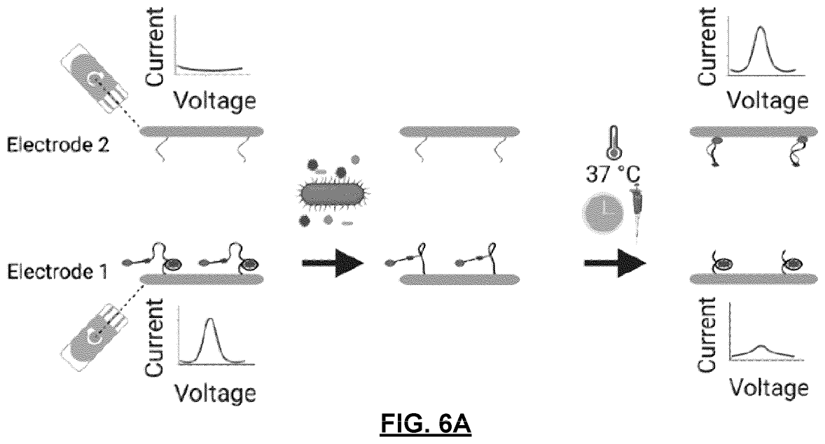

Peripheral Devices Validation: Solution heating is used in a broad range of biosensing experiments for sample preparation (e.g. lysis) and/or for expediting binding kinetics (e.g. DNA hybridization). To enable the translation of electrochemical biosensors with such requirements from the laboratory to the market, a portable heater, operated by the device, was created (FIG. 4A). To demonstrate the effectiveness of this sample heater, an on-chip electrode was heated, targeting 37° C., for 30 minutes. It was found that the surface temperature of the on-chip electrode reached 37° C. after approximately 15 minutes and plateaued at roughly 40° C. after 20 minutes (FIG. 4B). Other iterations of the sample heater may pre-heat the on-chip electrode in order to combat the long heating ramp time. The sample heater peripheral was further evaluated by using it in a DNA hybridization experiment (FIG. 4C). In this experiment, single-stranded probe DNA was immobilized on the surface of the working electrode. The target DNA sequence was tagged with methylene blue, a redox reporter. The hybridization of the immobilized probe with the redox DNA results in the generation of a signal. To detect DNA hybridization, square wave voltammetry was performed before and after target incubation. As previously discussed, data processing (Savitzky-Golay filtering and baseline subtraction) was conducted in-app. It was found that the heated on-chip electrodes produced a detectible signal; whereas those that were incubated at room temperature did not (FIG. 4D), demonstrating the effectiveness of the sample heater in expediting DNA hybridization.

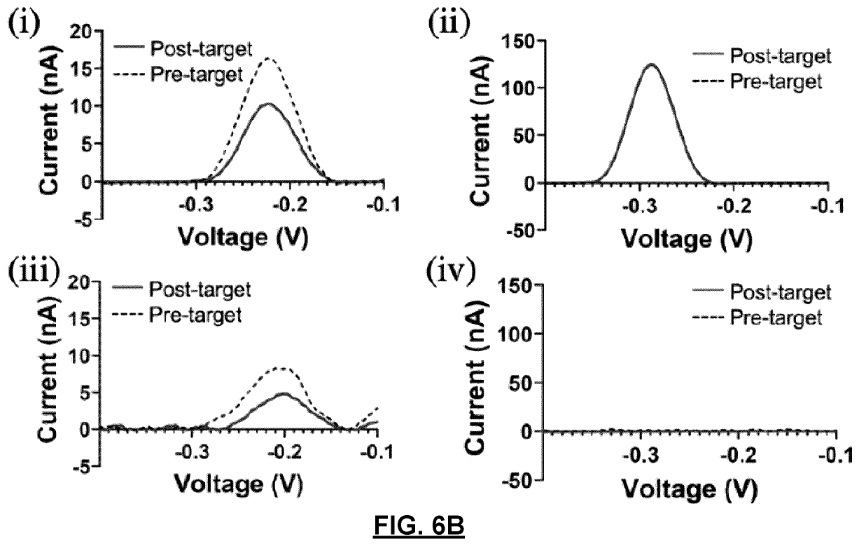

To showcase the device's magnetic manipulation capabilities, it was used to isolate magnetic microbeads in solution. The electromagnet was turned on and placed directly beside a rectangular vial filled with a solution of 14 μL of DI water and 1 μL of magnetic microbeads as shown in FIG. 5A for 30 minutes. This method adopted a specific orientation in order to highlight that the formation of the magnetic microbead congregation was not the result of gravitational forces. As shown in FIG. 5B and FIG. 5C respectively, photographs of the vial were taken before and after this experiment to showcase the formation of the magnetic microbead congregation.

Evaluation of a Two-Working Electrode Assay: Multiplexing is used in biological assays to evaluate a single sample for multiple target analytes and/or to obtain multiple readings per analyte for improved assay reliability. For such assays, it is necessary for the potentiostat to read out signals generated on multiple electrodes. To demonstrate the device's compatibility with multiplexed assays, it was used in conjunction with a two-working electrode assay. Briefly, this assay uses two working electrodes to detect E. coli. E. coli specific RNA-cleaving DNAzyme probes that are designed to cleave a segment of themselves in the presence of the target are surface-immobilized on the first electrode. Single-stranded DNA probes, which are designed to capture the cleaved segment of the DNAzyme, are surface-immobilized on the second electrode. In the presence of E. coli, the assay is designed to show a signal decrease on the first electrode and a signal increase on the second electrode.

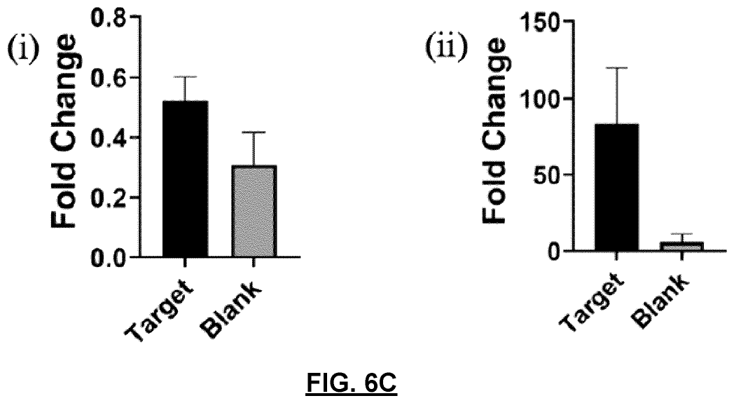

To validate the applicability of the device with the above-mentioned two-working electrode assay, square wave voltammograms were recorded at each stage of the assay operation (FIGS. 6A, 6B and 6C), using the MAX4644EUT to automatically toggle between the two electrodes. As expected, in the presence of E. coli, the peak current decreased on the first electrode and increased on the second electrode. For the blank solution, the signal still decreased on the first electrode, likely due to DNAzyme degradation; however, the signal changed by a much lesser amount on the second electrode in comparison to the target solution. The signal change on the two electrodes was calculated by subtracting the pre-target peak current from the post-target peak current and dividing the result by the pre-target peak current. In the event that no clear peaks were observed, as was the case with two of the first electrodes with target, the RMS noise values were used in place of the peak current. Data smoothening was not performed in these cases. On the first electrode, signal changes of 0.52 and 0.31 were observed for the target and blank samples, respectively, demonstrating a measurable difference (signal-to-blank ratio of 1.68) between the two samples. On the second electrode, much higher signal changes were measured using the target (84) and blank (6) samples, resulting in a remarkable signal-to-blank ratio of 14. Given the large signal changes on the second electrode, the data obtained from this electrode is more suitable for bioanalytical sensing. Nevertheless, the data obtained from the first electrode is critical for validating the quality of the manufactured chips.

### Example 2: Portable Photoelectrochemical Reader

### Materials and Methods

Caffeic acid (CA), phosphate buffered saline (PBS), L-ascorbic acid (AA), sodium hydroxide (NaOH), 1-ethyl-3-(3-dimethylaminopropyl)carbodiimide hydrochloride (EDC), N-hydroxysulfosuccinimide (NHS), 2-(N-morpholino)ethanesulfonic acid (MES), and 100 nm indium tin oxide coated poly(ethylene terephthalate) (ITO/PET) were purchased from Sigma-Aldrich. TiO₂ nanoparticles (P25, containing 80% anatase and 20% rutile) were obtained from Nippon Aerosil Co. Ltd. Anhydrous ethanol was purchased from Greenfield Global. Synthetic DNA sequences used capture probe and target were obtained from Integrated DNA Technologies.

Photoelectrode fabrication: An aqueous suspension of 0.66 g/L TiO₂ nanoparticles was prepared, to which a solution of CA dissolved in 5% ethanol in DI water solution was added to produce CA-surface modified TiO₂ nanoparticles (CA-TiO₂). The mass ratio of TiO₂ to CA in the CA-TiO₂ suspension was 10:1. ITO/PET substrates with dimensions of 1.2 cm×0.7 cm were masked with vinyl to preserve the electrical contact area. Substrates were subjected to (oxygen) plasma treatment for 1 min. The substrates were then coated with CA-TiO₂ by depositing 10 μL of the suspension on the substrate surface and incubating in the oven at 85° C. for 6 min. This process was repeated three times to deposit three layers.

Device design and preparation: For PEC biosensing, the portable biosensing readout system performs voltammetric techniques while synchronously controlling an LED matrix circuit which is used as an optical excitation source. This LED matrix circuit consists of four QT-Brightek PLCC6 white LEDs that have a neutral white color temperature (typically 4240K) and a typical intensity of 6000mcd. These LEDs have a maximum power rating of 324 mW and are driven by the 3.3V supply of the Arduino Nano 33 BLE. Printed circuit boards (PCB) for the portable biosensing readout system and the LED matrix circuit were designed using Eagle Autodesk and manufactured by JLCPCB based out of Shenzhen, Guangdong, China. The electronic components were then hand-soldered onto the PCBs. While many commercial potentiostats make use of multiple feedback resistors to adjust the current measurement range and sensitivity, to reduce device complexity, a single resistor was used. This yields a current measurement range of −10 μA to 10 μA and it was specifically in accordance with the photocurrent levels previously reported using the CA surface modified TiO₂ biosensor. The firmware was created using the Arduino integrated development environment. To facilitate debugging and access the serial monitor, the portable biosensing readout system was connected to a laptop running the Arduino IDE. The accompanying Android application was written in Java using the Android Studio integrated development environment.

Device characterization: A standard three-electrode cell setup was used with custom-made LED matrix circuit consisting of white light as a photoexcitation source. A platinum (Pt) wire was used as the counter electrode, a silver/silver chloride (Ag/AgCl) electrode for the reference electrode and the CA-TiO₂ photoelectrodes served as the working electrodes. The custom-made LED matrix circuit was used as an optical excitation source. The electrolyte solution used for the biasing experiment was composed of 0.5 M NaOH solution, whereas the electrolyte solution used for both the rapid PEC cycling and long-term exposure experiments was composed of 0.1 M PBS and 0.1 M AA. The photocurrent measurement was done by running the portable biosensing readout system in chronoamperometric mode. The potential of the PEC cell (DAC output) was fixed at a 0 V bias relative to an Ag/AgCl reference electrode, except for in the biasing experiment where the potential was varied. The performance of the portable biosensing readout system was compared to the Zahner CIMPS-QE/IPCE PEC workstation.

DNA hybridization and detection experiment: A solution of 20 mM EDC, 10 mM NHS, and 10 mM MES was deposited onto the photoelectrodes and incubated for 1 hour. This was done in order to facilitate carboxamide linking between CA and amine-terminated probe single-stranded DNA (ssDNA). Subsequently, 1 μM amine-terminated probe DNA was deposited on the photoelectrode surface and incubated for 2.5 hours. The photocurrent was measured at this point to get the after-probe photocurrent. For detecting DNA hybridization, the PEC signal was read out before and after incubation of the functionalized electrodes with a 100 nM target ssDNA solution for 1 hour. DNA hybridization detection was conducted using ssDNA-functionalized CA-TiO₂ photoelectrodes prepared using protocols described previously. Signal readout was performed using a PBS/AA electrolyte solution, and the LED matrix circuit as a photoexcitation source. When measuring the photocurrent of the PEC cell, the potential was kept constant at 0 V bias relative to an Ag/AgCl reference electrode and the working electrode was irradiated for 40 s in the middle of a 120 s runtime. To test nonspecific adsorption, some samples were spiked with 100 nM non-complementary ssDNA and/or 10% human blood plasma. The photoelectrodes were washed between each deposition step with deionized water. All the photocurrents were normalized by performing baseline subtraction of the dark current.

### Results and Discussion

Device design: a portable (75 mm by 40 mm, <100 g including a 3D printed case) and inexpensive (˜100 CAD) PEC reader was designed, which is wirelessly controlled by an Android application through the Bluetooth Low Energy (BLE) communication protocol to facilitate away-from-lab PEC experiments. This design capitalizes on the widespread global adoption of BLE-supported smartphones, rendering the portable biosensing readout system both smaller and less expensive than a standalone device. The specifications of the portable biosensing readout system is summarized in Table 3 below:

TABLE 3
Specifications of the portable biosensing readout system

| Current range | −10 μA to 10 μA | |
|---|---|---|
| | (−20 μA to 20 μA for differential measurements) | |
| Supply voltage | 4.5-21 | V |
| Signal generation | 16-bit (46 μV quantization error) | |
| resolution | | |
| ADC resolution | 24-bit | |
| Experimentalcurrent | 36 | pA |
| sensing resolution | | |
| Sampling rate | Programmable from 20 sps-1000 sps | |
| | (20 sps used in this work) | |
| Typical LED matrix | 4240 | K |
| color temperature | | |
| Typical LED matrix | 6000mcd | |
| intensity | | |

Alongside standard components that commonly make up a potentiostat (see, for example, in FIG. 7A)—a core processing unit (Arduino Nano 33 BLE development board), a digital-to-analog converter (DAC), a reconstruction filter, potentiostat circuitry consisting of a voltage follower (VF), control amplifier (CAmp), and transimpedance amplifier (TIA), and an analog-to-digital converter (ADC)—the portable biosensing readout system also interfaces with a peripheral LED matrix circuit (FIG. 7B) for PEC signal readout. The independence of the LED matrix circuit from the core device allows it to be modified (in terms of wavelength and intensity) to meet the optical requirements of different PEC assays.

A firmware function was developed to perform chronoamperometric scans while synchronously controlling the LED matrix circuit. This function makes use of timer interrupts to toggle the LED matrix circuit ON and OFF. Scan parameters for performing photoelectrochemical readout including the LED on and off time, the total duration of the scan, the voltage bias, pulse amplitude, pulse duration, and the sampling rate are all adjusted using the accompanying smartphone application. The smartphone application was designed to ensure it could easily be controlled by inexperienced users. By providing graphical animations, the Android application guides the user through the different stages of a typical PEC experiment (FIG. 7C). The collected scan measurements are then transmitted from the portable biosensing readout system to the smartphone via BLE for signal processing and to compile the results in a graphical format. In particular, a function was developed to perform in-app data smoothening using a Savitzky-Golay filter, an algorithm known for smoothening while preserving the shape and key features of the original signal.

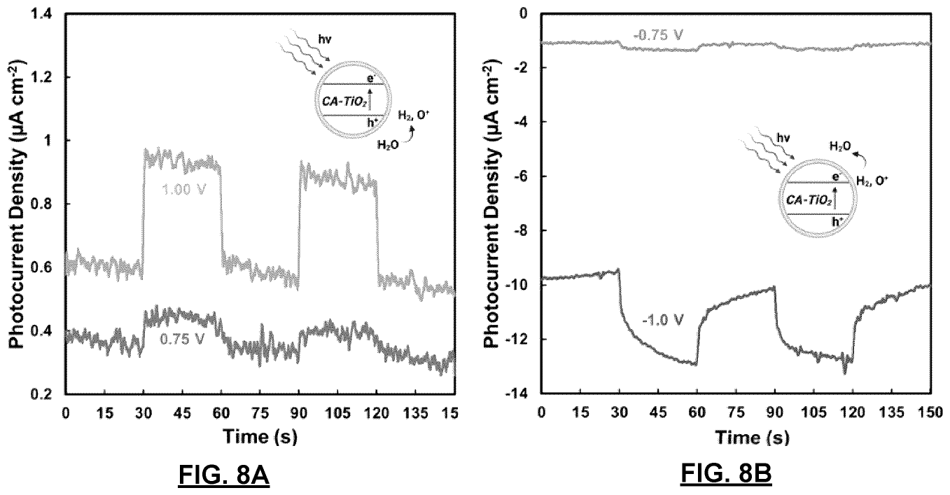

Device characterization: Experiments were conducted to demonstrate the versatility of the portable biosensing readout system and highlight its applicability for a wide array of PEC experiments. To demonstrate the ability of the portable biosensing readout system in measuring anodic and cathodic photocurrents, different bias voltages were applied (−1.0V, −0.75V, 0.75V, and 1.0V relative to an Ag/AgCl reference electrode) in an NaOH electrolyte solution and measured the resulting photocurrent. It was expected that the positive biases would yield anodic currents, and the negative biases would generate cathodic currents due to the oxidation of water and reduction of oxygen species, respectively. The working photoelectrodes, constructed via the drop deposition of TiO₂ nanoparticle suspensions onto a conductive polymer substrate, were irradiated with white light using the LED matrix circuit at 30 s intervals for 150 s, in order to generate the chronoamperometric curves (FIGS. 8A and 8B). As expected, the results demonstrated that the portable biosensing readout system could measure cathodic and anodic currents when positive and negative biases were applied. However, in the case of both the −0.75V and 0.75V biases (relative to an Ag/AgCl reference electrode), the signals were obfuscated by noise given their low amplitude. The noise can partly be attributed to the lack of shielding from background illumination and electrical interference. It is well known that the cathodic photocurrent generation of TiO₂ in alkaline solutions saturate in the region of −0.9 V to −1.0 V. This saturation is attributed to the diffusion limitation of oxygen species, thus limiting the reduction reaction. This explains the large increase in cathodic photocurrent from −0.75 V bias to −1.0 V bias (relative to an Ag/AgCl reference electrode), as the system has not yet reached saturation.

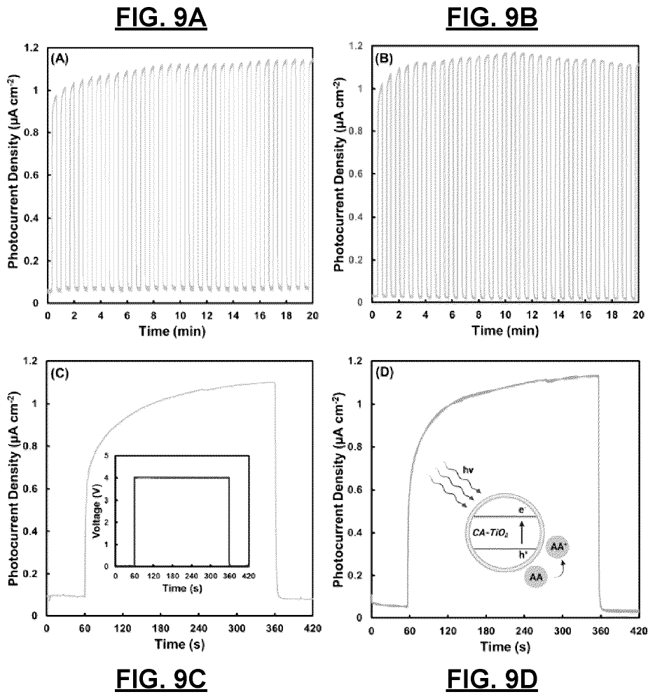

To further demonstrate the robustness of the portable biosensing readout system and showcase its applicability for PEC measurements with variable illumination periods, a PEC cycling test was conducted. The potential of the photoelectrode was held constant at 0 V bias relative to an Ag/AgCl reference electrode and concurrently irradiated with white light using the LED matrix circuit at 20-s intervals for 20 minutes (FIG. 9A). This experiment was performed using the same LED matrix circuit and the Zahner CIMPS-QE/IPCE PEC workstation, a benchtop measurement system used as a control (FIG. 9B). The two systems resulted in similar PEC currents that followed the illumination pattern, with gradual ramping of the photocurrents observed during the first 15 cycles. This behavior is owed to charge carriers getting trapped at the surface of the TiO₂ photoelectrode, producing a transient charging response. The short light-ON periods are not enough to reach the steady-state of the trap state induced transient response, and some discharge occurs at a slower rate during the light-OFF periods, resulting in the sawtooth photocurrent waveforms. The small decreases in photocurrent observed near the latter half of the experiment can be attributed to mechanical degradation of the photoelectrodes due to rapid cycling. The differences in the results obtained from the portable biosensing readout system and Zahner CIMPS-QE/IPCE PEC workstation, namely the larger photocurrent density recorded using the portable biosensing readout system during the LED OFF periods, can largely be attributed to the fact that the Zahner CIMPS-QE/IPCE PEC workstation was better shielded from background illumination and interference through the use of a faraday cage. This faraday cage was not used in conjunction with the portable biosensing readout system in order to better replicate the typical conditions associated with PoC measurements.

To investigate the electrical performance of the portable biosensing readout system, the input-referred noise was characterized. This parameter is a measure of the electrical noise induced exclusively by the circuitry and does not include any interference induced by the PEC cell. The working electrode connector was left open and readings were taken for 7 minutes. The ADC sampling rate was set to 20 sample-per-second, thereby enabling the built-in 50/60 Hz low-pass filter. This sampling rate is in line with all other measurements conducted in this work. The standard deviation of the open-circuit noise measurement was found to be 36 pA.

Next, a long-term light exposure test was performed in order to demonstrate that the LED matrix circuit could be used as a stable optical excitation source without significant fluctuations. Photocurrent measurements were conducted using a PBS/AA electrolyte solution. The AA electrolyte is a hole scavenger and its oxidation at the photoelectrode results in the generation of anodic photocurrent (FIG. 9D inset). The potential of the PEC cell was held constant at a 0 V bias relative to an Ag/AgCl reference electrode, and the working photoelectrode was irradiated with white light using the LED matrix circuit for 5 minutes, in order to generate the chronoamperometric curve (FIG. 9C). The performance was compared to the Zahner CIMPS-QE/IPCE PEC workstation (FIG. 9D). The expected anodic current was generated due to the oxidation of AA at the working electrode when under illumination (FIG. 3D inset). Additionally, the PEC curves measured using the portable biosensing readout system are nearly indistinguishable from those recorded by the Zahner CIMPS-QE/IPCE PEC workstation, featuring similar shapes and peak photocurrents. Using a photocell, the luminosity of the LED matrix circuit was measured over a 5-minute period (FIG. 9C inset) to verify consistent performance of the LED matrix circuit. Throughout this 5-minute period, the resistance of the photocell remained constant, indicating that the luminosity of the LED matrix circuit was stable. The photo-charging effect observed for the PEC cycling test is also present in the long-term exposure test. However, without the intermittent discharging, the photocurrent was able to come close to achieving steady-state within the 5-minute LED matrix circuit ON period.

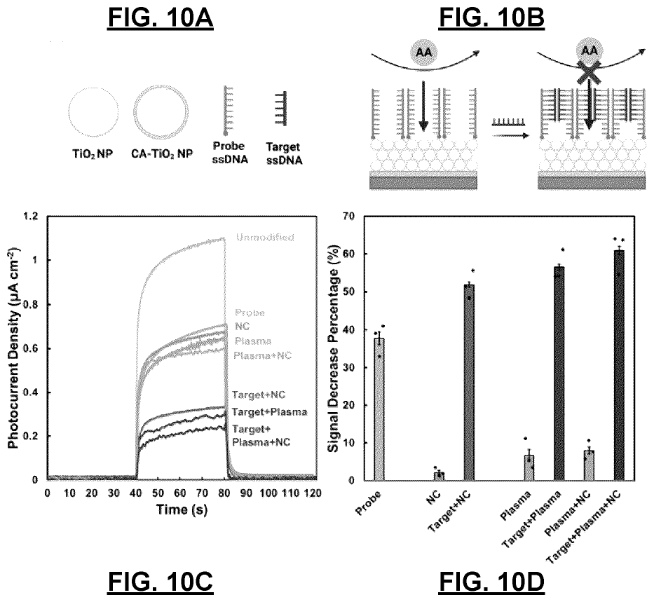

DNA hybridization and detection experiment: In order to demonstrate the applicability of the newly developed handheld platform for PEC biosensing, it was used to detect signal changes in a DNA hybridization detection assay. To this end, photoelectrodes were fabricated through drop deposition of TiO₂ nanoparticle suspensions onto a conductive polymer substrate. The unmodified photoelectrodes were composed of TiO₂ nanoparticles that were surface modified with CA to improve photocurrent generation by enhancing photo-absorption. The peak photocurrent density of the unmodified stage was 1.12 μA cm⁻². The surface of the photoelectrodes were modified with aminated single stranded DNA (ssDNA) capture probes by linking NH2 groups of the DNA to —COOH groups on the CA-modified TiO₂ nanoparticles (FIGS. 10A and 10B). DNA hybridization is expected to reduce the anodic current measured on the TiO₂ photoelectrodes by hindering the access of AA in the electrolyte to the electrode surface. To validate the performance of the device, the photocurrent density was measured after each stage of the assay development: unmodified; after-probe deposition; and after-target stages (FIG. 10C). Using this data, calculated the percent reduction was calculated in photocurrent relative to the previous stage (FIG. 10D). The deposition of 1 μM probe ssDNA resulted in a 37.7% average signal decrease. In the after-target stage, deposition of 100 nM non-complementary ssDNA in buffer (NC) as a control resulted in a slight 2.1% decrease in signal, which can attributed to non-specific DNA adsorption on the photoelectrode. In comparison, when a solution of 100 nM non-complementary and 100 nM complementary target ssDNA in buffer (Target+NC) was deposited, the observed signal reduction was much larger at 51.8%, due to DNA hybridization. The assay was challenged with samples of 10% human blood plasma with and without spiking of DNA. The blank samples containing only plasma (Plasma) and those containing 100 nM non-complementary ssDNA spiked in plasma (Plasma+NC) showed a signal decrease of 6.6% and 8.0%, respectively. The sample with 100 nM target complementary ssDNA in plasma (Target+Plasma) displayed a 56.5% signal reduction, whereas the sample with both 100 nM non-complementary ssDNA and 100 nM target complementary ssDNA spiked in plasma (Target+Plasma+NC) showed a higher signal reduction at 60.9%. While the protocol as presented in this work is slightly modified, these results are in-line with those previously reported by Sakib et al., which were recorded using a commercial PEC workstation. Accordingly, this demonstrates that the sensing abilities of the portable biosensing readout system is suitable for PEC DNA Biosensing at the PoC.

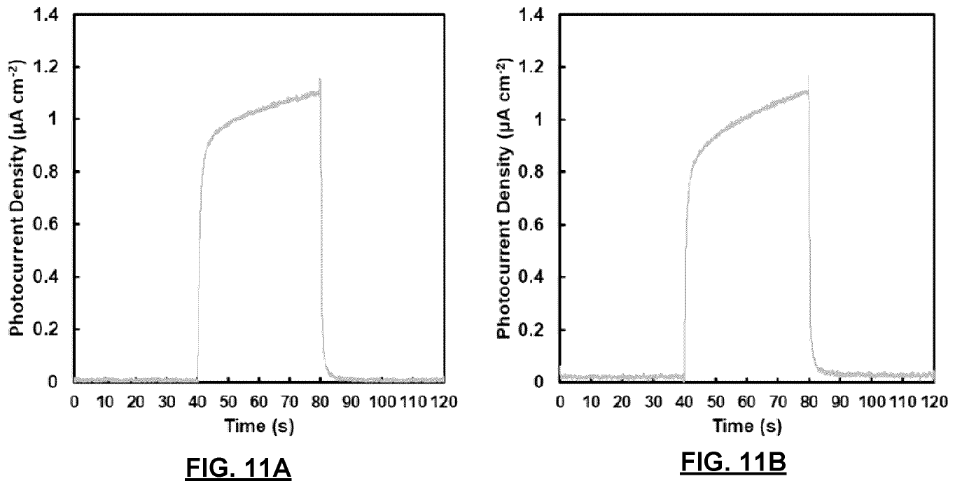

Illuminated chronoamperometry experiments: To further characterize the sensing abilities of the portable biosensing readout system, photocurrent measurements were conducted using bare CA-TiO₂ with and without the use of a Faraday cage for background signal shielding. As shown in FIGS. 11A and 11B, there were no significant differences between the two measurements. Although it is reasonable to assume that the elimination of background illumination would be advantageous in bright environments.

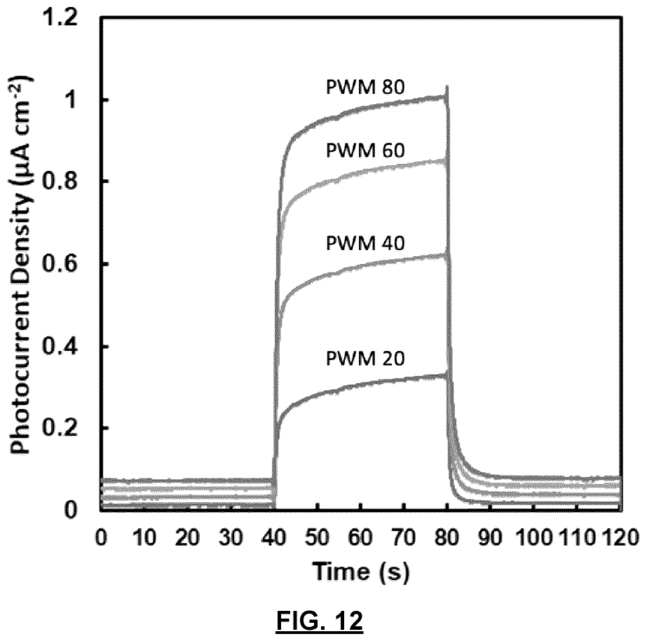

The LED matrix circuit is controlled via pulse width modulation (PWM) pins of the Arduino Nano 33 BLE, which operates at 500 Hz. By varying the PWM duty cycle (on/off time), the effective LED matrix circuit luminous intensity can be modified. As shown in FIG. 12, the peak photocurrent was found to be directly related to the duty cycle of the PWM signal.

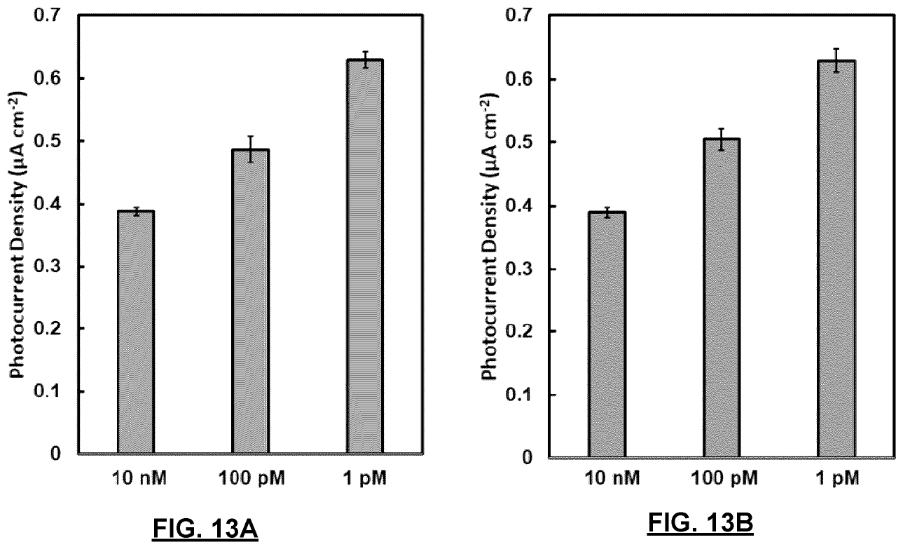

A partial limit of detection study was performed to highlight the portable biosensing readout system's capabilities in an analytical capacity. The portable biosensing readout system's ability to measure different target analyte concentrations was directly compared to the Zahner CIMPS-QE/IPCE PEC workstation, a commercially available potentiostat (as seen in FIGS. 13A and 13B). Target samples consisted of three different concentrations of single-stranded DNA (ssDNA) in buffer solution alongside 100 nM of non-complementary ssDNA to simulate the effects of non-specific absorption.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### REFERENCES

[1] L. Soleymani, F. Li, Mechanistic Challenges and Advantages of Biosensor Miniaturization into the Nanoscale, ACS Sensors. 2 (2017) 458-467. https://doi.org/10.1021/acssensors.7b00069.
[2] S. P. Mohanty, E. Koucianos, Biosensors: A tutorial review, IEEE Potentials. 25 (2006) 35-40. https://doi.org/10.1109/MP.2006.1649009.
[3] P. Mehrotra, Biosensors and their applications—A review, Journal of Oral Biology and Craniofacial Research. 6 (2016) 153-159. https://doi.org/10.1016/j.jobcr.2015.12.002.
[4] A. Victorious, S. Saha, R. Pandey, T. F. Didar, L. Soleymani, Affinity-Based Detection of Biomolecules Using Photo-Electrochemical Readout, Frontiers in Chemistry. 7 (2019). https://doi.org/10.3389/fchem.2019.00617.
[5] A. Hosseini, R. Pandey, E. Osman, A. Victorious, F. Li, T. Didar, L. Soleymani, Roadmap to the Bioanalytical Testing of COVID-19: From Sample Collection to Disease Surveillance, ACS Sensors. 5 (2020) 3328-3345. https://doi.org/10.1021/acssensors.0c01377.
[6] R. Pandey, D. Chang, M. Smieja, T. Hoare, Y. Li, L. Soleymani, Integrating programmable DNAzymes with electrical readout for rapid and culture-free bacterial detection using a handheld platform, Nature Chemistry. (2021). https://doi.org/10.1038/s41557-021-00718-x.
[7] A. P. Beltrán Lopez, DNA Biosensors and Biomarkers to Cancer Detection, International Journal of Biosensors & Bioelectronics. 4 (2018) 20-21. https://doi.org/10.15406/ijbsbe.2018.04.00090.
[8] S. M. Traynor, R. Pandey, R. Maclachlan, A. Hosseini, T. F. Didar, F. Li, L. Soleymani, Review—Recent Advances in Electrochemical Detection of Prostate Specific Antigen (PSA) in Clinically-Relevant Samples, Journal of The Electrochemical Society. 167 (2020) 037551. https://doi.org/10.1149/1945-7111/ab69fd.
[9] M. A. Khan, M. Mujahid, Recent advances in electrochemical and optical biosensors designed for detection of Interleukin 6, Sensors (Switzerland). 20 (2020) 1-27. https://doi.org/10.3390/s20030646.
[10] S. K. Metkar, K. Girigoswami, Diagnostic biosensors in medicine—A review, Biocatalysis and Agricultural Biotechnology. 17 (2019) 271-283. https://doi.org/10.1016/j.bcab.2018.11.029.
[11] V. Bianchi, M. Mattarozzi, M. Giannetto, A. Boni, I. de Munari, M. Careri, A self-calibrating iot portable electrochemical immunosensor for serum human epididymis protein 4 as a tumor biomarker for ovarian cancer, Sensors (Switzerland). 20 (2020). https://doi.org/10.3390/s20072016.
[12] K. R. King, L. P. Grazette, D. N. Paltoo, J. T. McDevitt, S. K. Sia, P. M. Barrett, F. S. Apple, P. A. Gurbel, R. Weissleder, H. Leeds, E. J. Iturriaga, A. K. Rao, B. Adhikari, P. Desvigne-Nickens, Z. S. Galis, P. Libby, Point-of-Care Technologies for Precision Cardiovascular Care and Clinical Research: National Heart, Lung, and Blood Institute Working Group, JACC: Basic to Translational Science. 1 (2016) 73-86. https://doi.org/10.1016/j.jacbts.2016.01.008.
[13] D. Harpaz, E. Eltzov, R. C. S. Seet, R. S. Marks, A. I. Y. Tok, Point-of-care-testing in acute stroke management: An unmet need ripe for technological Harvest, Biosensors. 7 (2017) 30. https://doi.org/10.3390/bios7030030.
[14] J. Quinchia, D. Echeverri, A. F. Cruz-Pacheco, M. E. Maldonado, J. A. Orozco, Electrochemical biosensors for determination of colorectal tumor biomarkers, Micromachines. 11 (2020) 1-46. https://doi.org/10.3390/MI11040411.
[15] E. Cesewski, B. N. Johnson, Electrochemical biosensors for pathogen detection, Biosensors and Bioelectronics. 159 (2020) 112214. https://doi.org/10.1016/j.bios.2020.112214.
[16] Yoo, E. H.; Lee, S. Y. Glucose Biosensors: An Overview of Use in Clinical Practice. Sensors 2010, 10 (5), 4558-4576. https://doi.org/i 0.3390/s100504558.
[17] Xie, H.; Di, K.; Huang, R.; Khan, A.; Xia, Y.; Xu, H.; Liu, C.; Tan, T.; Tian, X.; Shen, H.; He, N.; Li, Z. Extracellular Vesicles Based Electrochemical Biosensors for Detection of Cancer Cells: A Review. Chinese Chem. Lett. 2020, 31 (7), 1737-1745. https://doi.org/10.1016/j.cclet.2020.02.049.
[18] Diculescu, V. C.; Chiorcea-Paquim, A. M.; Oliveira-Brett, A. M. Applications of a DNA-Electrochemical Biosensor. TrAC—Trends in Analytical Chemistry. Elsevier B. V. May 1, 2016, pp 23-36. https://doi.org/10.1016/j.trac.2016.01.019.
[19] Liu, A.; Wang, K.; Weng, S.; Lei, Y.; Lin, L.; Chen, W.; Lin, X.; Chen, Y. Development of Electrochemical DNA Biosensors. TrAC—Trends Anal. Chem. 2012, 37, 101-111. https://doi.org/10.1016/j.trac.2012.03.008.
[20] Pfeiffer, F.; Mayer, G. Selection and Biosensor Application of Aptamers for Small Molecules. Front. Chem. 2016, 4 (JUN), 1-21. https://doi.org/10.3389/fchem.2016.00025.
[21] Hammond, J. L.; Formisano, N.; Estrela, P.; Carrara, S.; Tkac, J.

Electrochemical Biosensors and Nanobiosensors. Essays Biochem. 2016, 60 (1), 69-80. https://doi.org/10.1042/EBC20150008.
[22] S. Saha, A. Victorious, R. Pandey, A. Clifford, I. Zhitomirsky, L. Soleymani, Differential photoelectrochemical biosensing using DNA nanospacers to modulate electron transfer between metal and semiconductor nanoparticles, ACS Applied Materials and Interfaces. 12 (2020) 36895-36905. https://doi.org/10.1021/acsami.0c09443.
[23] S. Sakib, F. Bakhshandeh, S. Saha, L. Soleymani, I. Zhitomirsky, Surface Functionalization of Metal Oxide Semiconductors with Catechol Ligands for Enhancing Their Photoactivity, Solar RRL. n/a (n.d.) 2100512. https://doi.org/10.1002/solr.202100512.
[24] S. Sakib, R. Pandey, L. Soleymani, I. Zhitomirsky, Surface modification of TiO 2 for photoelectrochemical DNA biosensors, MEDICAL DEVICES & SENSORS. 3 (2020). https://doi.org/10.1002/mds3.10066.
[25] S. Roy, Z. Gao, Nanostructure-based electrical biosensors, Nano Today. 4 (2009) 318-334. https://doi.org/10.1016/j.nantod.2009.06.003.
[26] T. Chalklen, Q. Jing, S. Kar-Narayan, Biosensors based on mechanical and electrical detection techniques, Sensors (Switzerland). 20 (2020) 26-37. https://doi.org/10.3390/s20195605.
[27] J. X. Chen, Y. Zhuo, X. Peng, Y. Q. Chai, R. Yuan, W. bin Liang, A Dynamic DNA Machine via Free Walker Movement on Lipid Bilayer for Ultrasensitive Electrochemiluminescent Bioassay, Analytical Chemistry. 91 (2019) 14125-14132. https://doi.org/10.1021/acs.analchem.9b03999.
[28] P. Escobedo, M. M. Erenas, A. Martinez-Olmos, M. A. Carvajal, S. Gonzalez-Chocano, L. F. Capitán-Vallvéy, A. J. Palma, General-purpose passive wireless point-of-care platform based on smartphone, Biosensors and Bioelectronics. 141 (2019) 1-23. https://doi.org/10.1016/j.bios.2019.111360.
[29] J. L. Arlett, E. B. Myers, M. L. Roukes, Comparative advantages of mechanical biosensors, Nature Nanotechnology. 6 (2011) 203-215. https://doi.org/10.1038/nnano.2011.44.
[30] S. Vigneshvar, C. C. Sudhakumari, B. Senthilkumaran, H. Prakash, Recent advances in biosensor technology for potential applications—an overview, Frontiers in Bioengineering and Biotechnology. 4 (2016) 1-9. https://doi.org/1 0.3389/fbioe.2016.00011.
[31] E. Golub, G. Pelossof, R. Freeman, H. Zhang, I. Willner, Electrochemical, photoelectrochemical, and surface plasmon resonance detection of cocaine using supramolecular aptamer complexes and metallic or semiconductor nanoparticles, Analytical Chemistry. 81 (2009) 9291-9298. https://doi.org/10.1021/ac901551q.
[32] W. W. Zhao, J. J. Xu, H. Y. Chen, Photoelectrochemical Immunoassays, Analytical Chemistry. 90 (2018) 615-627. https://doi.org/10.1021/acs.analchem.7b04672.
[33] S. Saha, Y. Chan, L. Soleymani, Enhancing the Photoelectrochemical Response of DNA Biosensors Using Wrinkled Interfaces, ACS Applied Materials and Interfaces. 10 (2018) 31178-31185. https://doi.org/10.1021/acsami.8b12286.
[34] W. W. Zhao, J. J. Xu, H. Y. Chen, Photoelectrochemical DNA biosensors, Chemical Reviews. 114 (2014) 7421-7441. https://doi.org/10.1021/cr500100j.
[35] A. Devadoss, P. Sudhagar, C. Terashima, K. Nakata, A. Fujishima, Photoelectrochemical biosensors: New insights into promising photoelectrodes and signal amplification strategies, Journal of Photochemistry and Photobiology C: Photochemistry Reviews. 24 (2015) 43-63. https://doi.org/10.1016/j.jphotochemrev.2015.06.002.
[36] S. Sakib, A. Hosseini, I. Zhitomirsky, L. Soleymani, Photoelectrochemical IL-6 Immunoassay Manufactured on Multifunctional Catecholate-Modified TiO 2 Scaffolds, ACS Applied Materials & Interfaces. (2021). https://doi.org/10.1021/acsami.1c18240.
[37] Turner, A. P. F. Biosensors: Sense and Sensibility. Chem. Soc. Rev. 2013, 42 (8), 3184-3196. https://doi.org/10.1039/c3cs35528d.
[38] Palmsens. Sensit Smart—Palmsens.
[39] Adams, S. D.; Doeven, E. H.; Quayle, K.; Kouzani, A. Z. MiniStat: Development and Evaluation of a Mini-Potentiostat for Electrochemical Measurements. IEEE Access 2019. https://doi.org/10.1 109/ACCESS.2019.2902575.
[40] Bianchi, V.; Boni, A.; Fortunati, S.; Giannetto, M.; Careri, M.; De Munari, I. A Wi-Fi Cloud-Based Portable Potentiostat for Electrochemical Biosensors. IEEE Trans. Instrum. Meas. 2020, 69 (6), 3232-3240. https://doi.org/10.1109/TIM.2019.2928533.
[41] Ramfos, I.; Vassiliadis, N.; Blionas, S.; Efstathiou, K.; Fragoso, A.; O'Sullivan, C. K.; Birbas, A. A Compact Hybrid-Multiplexed Potentiostat for Real-Time Electrochemical Biosensing Applications. Biosens. Bioelectron. 2013, 47, 482-489. https://doi.org/10.1016/j.bios.2013.03.068.
[42] Irving, P.; Cecil, R.; Yates, M. Z. MYSTAT: A Compact Potentiostat/Galvanostat for General Electrochemistry Measurements.

HardwareX 2021, 9, e00163. https://doi.org/10.1016/j.ohx.2020.e00163.
[43] Krorakai, K.; Klangphukhiew, S.; Kulchat, S.; Patramanon, R. Smartphone-Based Nfc Potentiostat for Wireless Electrochemical Sensing. Appl. Sci. 2021, 11 (1), 1-13. https://doi.org/10.3390/app11010392.
[44] Barragan, J. T. C.; Kubota, L. T. Minipotentiostat Controlled by Smartphone on a Micropipette: A Versatile, Portable, Agile and Accurate Tool for Electroanalysis. Electrochim. Acta 2020, 341. https://doi.org/10.1016/j.electacta.2020.136048.
[45] Glasscott, M. W.; Verber, M. D.; Hall, J. R.; Pendergast, A. D.; McKinney, C. J.; Dick, J. E. SweepStat: A Build-It-Yourself, Two-Electrode Potentiostat for Macroelectrode and Ultramicroelectrode Studies. J. Chem. Educ. 2020, 97 (1), 265-270. https://doi.org/10.1021/acs.jchemed.9b00893.
[46] Giordano, G. F.; Vicentini, M. B. R.; Murer, R. C.; Augusto, F.; Ferrso, M. F.; Helfer, G. A.; da Costa, A. B.; Gobbi, A. L.; Hantao, L. W.; Lima, R. S. Point-of-Use Electroanalytical Platform Based on Homemade Potentiostat and Smartphone for Multivariate Data Processing. Electrochim. Acta 2016, 219, 170-177. https://doi.org/10.1016/j.electacta.2016.09.157.
[47] Dobbelaere, T.; Vereecken, P. M.; Detavernier, C. A USB-Controlled Potentiostat/Galvanostat for Thin-Film Battery Characterization. HardwareX 2017, 2, 34-49. https://doi.org/10.1016/j.ohx.2017.08.001.
[48] Dryden, M. D. M.; Wheeler, A. R. DStat: A Versatile, Open-Source Potentiostat for Electroanalysis and Integration. PloS One 2015, 10 (10). https://doi.org/10.1371/journal.pone.0140349.
[49] Ainla, A.; Mousavi, M. P. S.; Tsaloglou, M. N.; Redston, J.; Bell, J. G.; Fernendez-Abedul, M. T.; Whitesides, G. M. Open-Source Potentiostat for Wireless Electrochemical Detection with Smartphones. Anal. Chem. 2018, 90 (10), 6240-6246. https://doi.org/10.1021/acs.analchem.8b00850.
[50] Rowe, A. A.; Bonham, A. J.; White, R. J.; Zimmer, M. P.; Yadgar, R. J.; Hobza, T. M.; Honea, J. W.; Ben-Yaacov, I.; Plaxco, K. W. Cheapstat: An Open-Source, “Do-It-Yourself” Potentiostat for Analytical and Educational Applications. PloS One 2011, 6 (9). https://doi.org/10.1371/journal.pone.0023783.
[51] Fan, Y.; Liu, J.; Wang, Y.; Luo, J.; Xu, H.; Xu, S.; Cai, X. A Wireless Point-of-Care Testing System for the Detection of Neuron-Specific Enolase with Microfluidic Paper-Based Analytical Devices. Biosens. Bioelectron. 2017, 95 (April), 60-66. https://doi.org/10.1016/j.bios.2017.04.003.
[52] Jenkins, D. M.; Lee, B. E.; Jun, S.; Reyes-De-Corcuera, J.; McLamore, E. S. ABE-Stat, a Fully Open-Source and Versatile Wireless Potentiostat Project Including Electrochemical Impedance Spectroscopy. J. Electrochem. Soc. 2019, 166 (9), B3056-B3065. https://doi.org/10.1149/2.0061909jes.
[53] Jafari, H. M.; Genov, R. Chopper-Stabilized Bidirectional Current Acquisition Circuits for Electrochemical Amperometric Biosensors. IEEE Trans. Circuits Syst. I Regul. Pap. 2013, 60 (5), 1149-1157. https://doi.org/10.1109/TCSI.2013.2248771.
[54] Shen, X.; Ju, F.; Li, G.; Ma, L. Smartphone-Based Electrochemical Potentiostat Detection System Using Pedot: Pss/Chitosan/Graphene Modified Screen-Printed Electrodes for Dopamine Detection. Sensors (Switzerland) 2020, 20 (10). https://doi.org/10.3390/s20102781.
[55] Cordova-Huaman, A. V.; Jauja-Ccana, V. R.; La Rosa-Toro, A. Low-Cost Smartphone-Controlled Potentiostat Based on Arduino for Teaching Electrochemistry Fundamentals and Applications. Heliyon 2021, 7 (2), e06259. https://doi.org/10.1016/j.heliyon.2021.e06259.
[56] Sepúlveda, F.; Hernández, J.; Manríquez, A. Construction of a Potentiostat to Perform Electrochemical Impedance Spectroscopy (EIS) Tests. Mod. Instrum. 2017, 06 (02), 15-27. https://doi.org/10.4236/mi.2017.62002.
[57] Ji, D.; Liu, L.; Li, S.; Chen, C.; Lu, Y.; Wu, J.; Liu, Q. Smartphone-Based Cyclic Voltammetry System with Graphene Modified Screen Printed Electrodes for Glucose Detection. Biosens. Bioelectron. 2017, 98 (March), 449-456. https://doi.org/10.1016/j.bios.2017.07.027.
[58] Hoilett, O. S.; Walker, J. F.; Balash, B. M.; Jaras, N. J.; Boppana, S.; Linnes, J. C. Kickstat: A Coin-Sized Potentiostat for High-Resolution Electrochemical Analysis. Sensors (Switzerland) 2020, 20 (8), 1-12. https://doi.org/i 0.3390/s20082407.
[59] Siegert, M. A Scalable Multi-Channel Software Potentiostat. Front. Energy Res. 2018, 6 (November), 1-4. https://doi.org/10.3389/fenrg.2018.00131.
[60] Palec̆ek, E.; Fojta, M. Magnetic Beads as Versatile Tools for Electrochemical DNA and Protein Biosensing. Talanta 2007, 74 (3), 276-290. https://doi.org/10.1016/j.talanta.2007.08.020.
[61] T. Yagura, K. Makita, H. Yamamoto, C. F. M. Menck, A. P. Schuch, Biological sensors for solar ultraviolet radiation, Sensors (Switzerland). 11 (2011) 4277-4294. https://doi.org/10.3390/s110404277.
[62] Z. Han, M. Luo, L. Chen, H. Pan, J. Chen, C. Li, A photoelectrochemical biosensor for determination of DNA based on flower rod-like zinc oxide heterostructures, Microchimica Acta. 184 (2017) 2541-2549. https://doi.org/i 0.1007/s00604-017-2257-5.
[63] X. Zhang, Y. Zhao, S. Li, S. Zhang, Photoelectrochemical biosensor for detection of adenosine triphosphate in the extracts of cancer cells, Chemical Communications. 46 (2010) 9173-9175. https://doi.org/10.1039/c0cc03595e.
[64] W. Wang, L. Bao, J. Lei, W. Tu, H. Ju, Visible light induced photoelectrochemical biosensing based on oxygen-sensitive quantum dots, Analytica Chimica Acta. 744 (2012) 33-38. https://doi.org/10.1016/j.aca.2012.07.025.
[65] X. Zhang, F. Xu, B. Zhao, X. Ji, Y. Yao, D. Wu, Z. Gao, K. Jiang, Synthesis of CdS quantum dots decorated graphene nanosheets and non-enzymatic photoelectrochemical detection of glucose, Electrochimica Acta. 133 (2014) 615-622. https://doi.org/10.1016/j.electacta.2014.04.089.
[66] Z. Yue, F. Lisdat, W. J. Parak, S. G. Hickey, L. Tu, N. Sabir, D. Dorfs, N. C. Bigall, Quantum-dot-based photoelectrochemical sensors for chemical and biological detection, ACS Applied Materials and Interfaces. 5 (2013) 2800-2814. https://doi.org/10.1021/am3028662.
[67] X. Zhang, Y. Xu, Y. Yang, X. Jin, S. Ye, S. Zhang, L. Jiang, A new signal-on photoelectrochemical biosensor based on a graphene/quantum-dot nanocomposite amplified by the dual-quenched effect of bipyridinium relay and AuNPs, Chemistry—A European Journal. 18 (2012) 16411-16418. https://doi.org/10.1002/chem.201202213.
[68] Z. Li, J. Zhang, Y. Li, S. Zhao, P. Zhang, Y. Zhang, J. Bi, G. Liu, Z. Yue, Carbon dots based photoelectrochemical sensors for ultrasensitive detection of glutathione and its applications in probing of myocardial infarction, Biosensors and Bioelectronics. 99 (2018) 251-258. https://doi.org/10.1016/j.bios.2017.07.065.
[69] H. Gergeroglu, S. Yildirim, M. F. Ebeoglugil, Nano-carbons in biosensor applications: an overview of carbon nanotubes (CNTs) and fullerenes (C60), SN Applied Sciences. 2 (2020). https://doi.org/10.1007/s42452-020-2404-1.
[70] B. S̆pac̆ková, P. Wrobel, M. Bocková, J. Homola, Optical Biosensors Based on Plasmonic Nanostructures: A Review, Proceedings of the IEEE. 104 (2016) 2380-2408. https://doi.org/10.1109/JPROC.2016.2624340.
[71] A. Kongkanand, R. M. Domiinguez, P. v. Kamat, Single Wall Carbon Nanotube Scaffolds for Photoelectrochemical Solar Cells. Capture and Transport of Photogenerated Electrons, Nano Letters. 7 (2007) 676-680. https://doi.org/1 0.3141/1528-08.
[72] C. Han, Y. H. Li, M. Y. Qi, F. Zhang, Z. R. Tang, Y. J. Xu, Surface/Interface Engineering of Carbon-Based Materials for Constructing Multidimensional Functional Hybrids, Solar RRL. 4 (2020) 1-25. https://doi.org/10.1002/solr.201900577.
[73] I. Arbouch, D. Cornil, Y. Karzazi, B. Hammouti, R. Lazzaroni, J. Cornil, Influence of the nature of the anchoring group on electron injection processes at dye-titania interfaces, Physical Chemistry Chemical Physics. 19 (2017) 29389-29401. https://doi.org/10.1039/c7cp05638a.
[74] G. C. Fan, X. L. Ren, C. Zhu, J. R. Zhang, J. J. Zhu, A new signal amplification strategy of photoelectrochemical immunoassay for highly sensitive interleukin-6 detection based on TiO2/CdS/CdSe dual co-sensitized structure, Biosensors and Bioelectronics. 59 (2014) 45-53. https://doi.org/10.1016/j.bios.2014.03.011.
[75] S. Sarkar, A. Makhal, K. Lakshman, T. Bora, J. Dutta, S. Kumar Pal, Dual-sensitization via electron and energy harvesting in CdTe quantum dots decorated ZnO nanorod-based dye-sensitized solar cells, Journal of Physical Chemistry C. 116 (2012) 14248-14256. https://doi.org/10.1021/jp3046593.
[76] Palmsens, Palmsens 4 Palmsens, (2021). https://www.palmsens.com/product/palmsens4/(accessed Sep. 18, 2021).
[77] Q. Zhang, Z. Chen, Z. Shi, Y. Li, Z. An, X. Li, J. Shan, Y. Lu, Q. Liu, Smartphone-based photoelectrochemical biosensing system with graphitic carbon nitride/gold nanoparticles modified electrodes for matrix metalloproteinase-2 detection, Biosensors and Bioelectronics. 193 (2021) 113572. https://doi.org/10.1016/j.bios.2021.113572.
[78] A. Sun, T. Wambach, A. G. Venkatesh, D. A. Hall, A low-cost smartphone-based electrochemical biosensor for point-of-care diagnostics, in: IEEE 2014 Biomedical Circuits and Systems Conference, BioCAS 2014—Proceedings, 2014. https://doi.org/10.1109/BioCAS.2014.6981725.
[79] A. C. Sun, D. A. Hall, Point-of-Care Smartphone-based Electrochemical Biosensing, Electroanalysis. 31 (2019) 2-16. https://doi.org/10.1002/elan.201800474.
[80] van Welie, M. BLESSED BLE. 2021.
[81] Jahoda, P. MPAndroidChart. 2021.
[82] Rzeźnicki, M. SGFilter. 2021.
[83] Ali, M. M.; Brown, C. L.; Jahanshahi-Anbuhi, S.; Kannan, B.; Li, Y.; Filipe, C. D. M.; Brennan, J. D. A Printed Multicomponent Paper Sensor for Bacterial Detection. Sci. Rep. 2017, 7 (1), 1-10. https://doi.org/10.1038/s41598-017-12549-3.
[84] Colburn, A. W.; Levey, K. J.; O'Hare, D.; Macpherson, J. V. Lifting the Lid on the Potentiostat: A Beginner's Guide to Understanding Electrochemical Circuitry and Practical Operation. Phys. Chem. Chem. Phys. 2021, 23 (14), 8100-8117. https://doi.org/10.1039/d1cp00661d.
[85] Ju, H. Signal Amplification for Highly Sensitive Bioanalysis Based on Biosensors or Biochips. J. Biochips Tissue Chips 2012, 02 (02), 2-3. https://doi.org/1 0.4172/2153-0777.1000e 114.
[86] Acharya, D.; Rani, A.; Agarwal, S.; Singh, V. Application of Adaptive Savitzky-Golay Filter for EEG Signal Processing. Perspect. Sci. 2016, 8, 677-679. https://doi.org/10.1016/j.pisc.2016.06.056.
[87] Feuerstein, D.; Parker, K. H.; Boutelle, M. G. Practical Methods for Noise Removal: Applications to Spikes, Nonstationary Quasi-Periodic Noise, and Baseline Drift. Anal. Chem. 2009, 81 (12), 4987-4994. https://doi.org/10.1021/ac900161x.
[88] Gonçalves, W. D.; Lanfredi, A. J. C.; Crespilho, F. N. Development of Numerical Methods for Signal Smoothing and Noise Modeling in Single Wire-Based Electrochemical Biosensors. J. Phys. Chem. C 2011, 115 (32), 16172-16179. https://doi.org/10.1021/jp204180e.
[89] Górski, L; Ciepiela, F.; Jakubowska, M. Automatic Baseline Correction in Voltammetry. Electrochim. Acta 2014, 136, 195-203. https://doi.org/10.1016/j.electacta.2014.05.076.
[90] Bard, A. J.; Faulkner, L. R. Electrochemical Methods: Fundamentals and Applications, 2nd ed.; Wiley-Interscience: New York, New York, United States, 2000.
[91] Billings, P. R. Three Barriers to Innovative Diagnostics. Nat. Biotechnol. 2006, 24 (8), 917-918. https://doi.org/10.1038/nbt0806-917.
[92] Ren, K.; Wu, J.; Yan, F.; Ju, H. Ratiometric Electrochemical Proximity Assay for Sensitive One-Step Protein Detection. Sci. Rep. 2014, 4, 1-6. https://doi.org/10.1038/srep04360.
[93] Aidoo-Brown, J.; Moschou, D.; Estrela, P. Multiplexed Prostate Cancer Companion Diagnostic Devices. Sensors 2021, 21 (15). https://doi.org/1 0.3390/s21155023.
[94] Tang, Z.; Ma, Z. Ratiometric Ultrasensitive Electrochemical Immunosensor Based on Redox Substrate and Immunoprobe. Sci. Rep. 2016, 6 (September), 2-7. https://doi.org/10.1038/srep35440.
[95] Yáñez-Sedeño, P.; Campuzano, S.; Pingarrón, J. M. Multiplexed Electrochemical Immunosensors for Clinical Biomarkers. Sensors (Switzerland) 2017, 17 (5). https://doi.org/10.3390/s17050965.
[96] Liu, M.; Zhang, Q.; Brennan, J. D.; Li, Y. Graphene-DNAzyme-Based Fluorescent Biosensor for Escherichia Coli Detection. MRS Commun. 2018, 8 (3), 687-694. https://doi.org/10.1557/mrc.2018.97.
[97] Zhang, J. RNA-Cleaving DNAzymes: Old Catalysts with New Tricks for Intracellular and in Vivo Applications. Catalysts 2018, 8 (11), 1-20. https://doi.org/1 0.3390/catal8110550.
[98] A. Tsujiko, H. Itoh, T. Kisumi, A. Shiga, K. Murakoshi, Y. Nakato, Observation of cathodic photocurrents at nanocrystalline TiO2 film electrodes, caused by enhanced oxygen reduction in alkaline solutions, Journal of Physical Chemistry B. 106 (2002) 5878-5885. https://doi.org/10.1021/jp0l21441.
[99] M. Dumortier, T. Bosserez, J. Rongé, J. A. Martens, S. Haussener, Combined Experimental-Numerical Analysis of Transient Phenomena in a Photoelectrochemical Water Splitting Cell, Journal of Physical Chemistry C. 120 (2016) 3705-3714. https://doi.org/10.1021/acs.jpcc.5b12445.
[100] Z. Liu, W. Hou, P. Pavaskar, M. Aykol, S. B. Cronin, Plasmon resonant enhancement of photocatalytic water splitting under visible illumination, Nano Letters. 11 (2011) 1111-1116. https://doi.org/10.1021/nl104005n.
[101] Y. Li, Y. Xu, C. C. Fleischer, J. Huang, R. Lin, L. Yang, H. Mao, Impact of anti-biofouling surface coatings on the properties of nanomaterials and their biomedical applications, Journal of Materials Chemistry B. 6 (2017) 9-24. https://doi.org/10.1039/c7tb01695f.

1. A portable biosensing readout system for analyzing a sample, the portable biosensing readout system comprising:
a biosensor comprising a working electrode, a reference electrode and a counter electrode;
a core potentiostat circuit coupled to the biosensor, the core potentiostat circuit having one or more amplifiers configured to:
inject an excitation signal into the biosensor;
isolate the reference electrode; and
convert current of an output signal from the biosensor to voltage;
a microcontroller unit in communication with the core potentiostat circuit, the microcontroller unit being configured to:
generate various different types of excitation signals that are transmitted to the core potentiostat circuit and, subsequently, to the biosensor;
receive the output signal from the core potentiostat circuit; and
communicate the output signal to an external computing device; and
a peripheral instrument for processing the sample, the peripheral instrument being in communication with the microcontroller unit to receive instructions from the microcontroller that control the peripheral instrument.

2. The portable biosensing readout system of claim 1, wherein the peripheral instrument is one or more of a heater, a magnetic module, an electromagnet and one or more tunable light sources.

3. The portable biosensing readout system of claim 1, wherein the output signal from the biosensor is an electrochemical signal or a photoelectrochemical signal.

4. The portable biosensing readout system of claim 1, wherein the microcontroller unit comprises an Arduino device that processes the output from the core potentiostat circuit.

5. The portable biosensing readout system of claim 1, wherein the microcontroller unit is coupled to a digital-to-analog converter and a reconstruction filter.

6. (canceled)

7. The portable biosensing readout system of claim 5, wherein the microcontroller unit is further configured to:
generate a voltammetric excitation signal;
compute a voltammetric excitation series;
read the output signal received from the core potentiostat circuit;
configure settings of various integrated circuits if the biosensing readout system; and
communicate with the external computing device.

8. The portable biosensing readout system of claim 1, wherein the portable biosensing readout system further comprises:
a dual output voltage reference;
a multiplexer; and
an analog-to-digital converter; and
the plurality of amplifiers includes a control amplifier, a voltage follower, and a transimpedance amplifier.

9. The portable biosensing readout system of claim 8, wherein the multiplexer is configured to toggle between two or more working electrodes to perform sequential measurements of dual-signal assays or multi-channel assays.

10. (canceled)

11. (canceled)

12. The portable biosensing readout system of claim 8, wherein the plurality of amplifiers further comprises a dual operational amplifier, a precision operational amplifier, or a combination thereof.

13. (canceled)

14. (canceled)

15. (canceled)

16. The portable biosensing readout system of claim 8, wherein the core potentiostat circuit includes a dual impedance converter/network analyzer chip.

17. The portable biosensing readout system of claim 8, wherein the core potentiostat circuit is configured to perform multiplexed measurements.

18. The portable biosensing readout system of claim 1, wherein the biosensor includes a biorecognition moiety immobilized onto a surface of the working electrode to recognize a presence of an analyte in the sample, the biorecognition moiety comprising a DNAzyme, an aptamer, an antibody, a nucleic acid, or a combination thereof.

19. The portable biosensing readout system of claim 1, wherein the peripheral instrument is a heater configured to heat the sample or a magnetic module configured to perform magnetic manipulation of the sample.

20. (canceled)

21. The portable biosensing readout system of claim 1, wherein the peripheral instrument is an electromagnet configured to perform electromagnetic manipulation of the sample.

22. (canceled)

23. The portable biosensing readout system of claim 1, wherein the peripheral instrument is a light source configured to optically excite the sample, the light source comprising a light emitting diode (LED) matrix circuit comprising one or more LEDs.

24. (canceled)

25. (canceled)

26. (canceled)

27. The portable biosensing readout system of claim 23, wherein the LED matrix circuit has modifiable parameters for different assays, the modifiable parameters including on and off time and optical parameters, the optical parameters including LED intensity.

28. The portable biosensing readout system of claim 1, wherein the analyzing of the sample is by square wave voltammetry, cyclic voltammetry, linear sweep voltammetry, differential pulse voltammetry, normal pulse voltammetry, electrochemical impedance spectroscopy, chronoamperometry, or a combination thereof.

29. The portable biosensing readout system of claim 1, wherein the microcontroller unit is configured to control a total duration of a scan of the sample, control a voltage bias of the excitation signal, control a pulse amplitude of the excitation signal, control a pulse duration of the excitation signal and/or control a sampling rate of the excitation signal.

30. (canceled)

31. (canceled)

32. (canceled)

33. (canceled)

34. (canceled)

35. A method of analyzing a sample by a portable biosensing readout system, the method comprising:
selecting one or more peripheral instruments to attach to a portable biosensing readout system;
attaching the one or more peripheral instruments to the portable biosensing readout system;
contacting the portable biosensing readout system with the sample to detect an output signal; and
transmitting the output signal as data to an external computing device; and
analyzing the data by the external computing device.

36. (canceled)

37. (canceled)

38. The method of claim 35, wherein a biorecognition moiety on a biosensor of the portable biosensing readout system comprises DNAzymes, aptamers, antibodies, nucleic acids, or a combination thereof that is configured to recognize the presence of an analyte in the sample.
